(19) United States
(12) Patent Application Publication
(10) Pub. No.: US 2024/0009208 A1
(43) Pub. Date: Jan. 11, 2024

(54) STABILIZATION OF CANNABINOIDS WITHIN SOLID DOSAGE FORMS

(71) Applicant: RECEPTOR HOLDINGS, INC., Seattle, WA (US)

(72) Inventors: Kelly Kraft, Poughquag, NY (US); Mark Hokenson, Camarillo, CA (US)

(73) Assignee: RECEPTOR HOLDINGS, INC., Seattle, WA (US)

(21) Appl. No.: 18/247,417

(22) PCT Filed: Oct. 1, 2021
(86) PCT No.: PCT/US2021/053220

§ 371 Date: Mar. 30, 2023

Related U.S. Application Data
(60) Provisional application No. 63/087,029, filed on Oct. 2, 2020.

(51) Int. Cl. A61K 31/00; A61K 9/00; A61K 9/16

(52) U.S. Cl. CPC A61K 31/658; A61K 9/0075; A61K 9/1617; A61K 9/1623; A61K 9/1694

(57) ABSTRACT

The use of phosphatidylcholines to directly stabilize cannabinoids within dry powder is described. Phosphatidylcholines include 1,2-distearoyl-sn-glycero-3-phosphocholine (DSPC) and 1,2-dipalmitoyl-sn-glycero-3-phosphocholine (DPPC) and cannabinoids include tetrahydrocannabinol (THC) and cannabidiol (CBD). The dry powder containing the stabilized cannabinoids can be formulated for inhalation or oral delivery. Dry powder formulated for inhalation can include formyl diketopiperazine (FDKP). Oral delivery of the dry powder containing the stabilized cannabinoids includes delivery to the gastrointestinal tract and to the mucous membranes of the oral cavity.

### CROSS-REFERENCE TO RELATED APPLICATION

This application is a U.S. National Phase Patent Application based on International Patent Application No. PCT/US2021/053220, filed on Oct. 1, 2021, which claims priority to U.S. Provisional Patent Application No. 63/087,029, filed on Oct. 2, 2020, each of which is incorporated herein by reference in its entirety as if fully set forth herein.

### FIELD OF THE DISCLOSURE

The current disclosure describes use of phosphatidylcholines to directly stabilize cannabinoids within solid dosage forms, such as dry powders for inhalation or tablet or capsule formation. Phosphatidylcholines include 1,2-distearoyl-sn-glycero-3-phosphocholine (DSPC) and 1,2-dipalmitoyl-sn-glycero-3-phosphocholine (DPPC) and cannabinoids include tetrahydrocannabinol (THC) and cannabidiol (CBD).

### BACKGROUND OF THE DISCLOSURE

Cannabinoids are a diverse class of compounds that interact with and activate cannabinoid receptors. There are three classes of cannabinoids: 1) endocannabinoids, which are naturally produced in the body by humans and other animals, 2) phytocannabinoids, which are produced by plants, and 3) synthetic cannabinoids, which are chemically produced cannabinoids. Synthetic cannabinoids can be identical to cannabinoids that are found in nature or can be compounds that do not exist in nature.

(−)-trans-delta-9-tetrahydrocannabinol (THC, Δ⁹-THC) is the most extensively studied cannabinoid and has many well-established health benefits. THC is prescribed under the pharmaceutical drug name dronabinol, and is FDA approved for use as an appetite stimulant for HIV- and AIDS-related weight loss and for chemotherapy-induced nausea and vomiting. Many other medical uses of THC are being investigated, and research indicates that THC may have anti-tumor activity (Guzman M, Nat Rev Cancer. 2003. 3:745-55), anti-inflammatory effects (Gaiffal E, et al. Allergy. 2013. 68(8): 994-1000), and analgesic effects (Pharm. J. 259, 104, 1997 and in Pharm. Sci. 3, 546, 1997).

Nabilone, a synthetic cannabinoid not found in nature, is another cannabinoid that has numerous medical uses. Nabilone, which is structurally very similar to THC, has been reported to be an anti-emetic and anxiolytic, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7).

Another cannabinoid with well-documented health benefits is cannabidiol (CBD). In contrast to THC, CBD does not exert psychoactive effects. CBD is reported to have antidepressant (Zanelati T, et al. Journal of Pharmacology. 2010. 159(1):122-8;), anti-anxiety (Resstel B M, et al. Br J Pharmacol. 2009. 156(1):181-188), anti-inflammatory (Vuolo F, et al. Mediators of Inflammation. 2015. 538670), and neuroprotective effects (Campos A C, et al. Pharmacol Res. 2016. 112:119-127).

While cannabinoids have numerous health benefits, challenges to their formulation arise due to issues surrounding their stability during storage. For example, THC extracts are generally stored under refrigerated conditions and/or under inert conditions (e.g., stored under nitrogen) to improve the shelf life (chemical stability) of the THC. These storage requirements add significant expense and logistics to the storage of THC-based compositions. Moreover, while cannabinoids generally show increased stability when diluted into liquid formulations, a need for stabilized cannabinoids within solid dosage forms remains.

### SUMMARY OF THE DISCLOSURE

The current disclosure describes use of phosphatidylcholines (PC) to directly stabilize cannabinoids within solid dosage forms, such as dry powders for inhalation or tablet or capsule formulations. Exemplary PC include 1,2-distearoyl-sn-glycero-3-phosphocholine (DSPC) and 1,2-dipalmitoyl-sn-glycero-3-phosphocholine (DPPC). Exemplary cannabinoids include tetrahydrocannabinol (THC) and cannabidiol (CBD). As disclosed herein, selected PC stabilize cannabinoids within solid dry powders for at least 32 weeks. In particular embodiments, selected PC stabilize cannabinoids within solid dry powders for at least 63 weeks. Particular embodiments include solid dosage forms with 5-30% PC and 5-50% cannabinoids by weight. More particular embodiments include solid dosage forms with 5-20% PC and 10-30% cannabinoids by weight. When formulated as an inhalation dry powder, the solid dosage forms can include fumaryl diketopiperazine (FDKP). When formulated as a tablet or capsule for oral administration, the solid dosage forms can include an N-acylated fatty amino acid to increase the bioavailability of the cannabinoids following administration.

### BRIEF DESCRIPTION OF THE SEVERAL VIEWS OF THE DRAWINGS

FIG. 1. shows the stability of 26% w/w tetrahydrocannabinol (THC) inhalation powders with varying percentages of 1,2-distearoyl-sn-glycero-3-phosphocholine (DSPC) expressed as % w/w of the inhalation powder: 5% DSPC; 10% DSPC; 15% DSPC; 20% DSPC; 25% DSPC; and 30% DSPC.

FIG. 2. shows the stability of 26% w/w THC inhalation powders with varying percentages of 1,2-dipalmitoyl-sn-glycero-3-phosphocholine (dipalmitoylphosphatidylcholine; DPPC) expressed as % w/w of the inhalation powder: 10% DPPC; 15% DPPC; 20% DPPC and 25% DPPC.

FIG. 3. shows the stability of 10% w/w THC inhalation powders with varying percentages of DSPC expressed as % w/w of the inhalation powder. (F1) stability of a 10% w/w THC inhalation powder with no DSPC added; (F2) stability of a 10% w/w THC, 5% w/w DSPC inhalation powder; and (F3) stability of a 10% w/w THC, 20% w/w DSPC inhalation powder.

FIG. 4. shows the stability of 20% w/w THC inhalation powders with varying percentages of DSPC expressed as % w/w of the inhalation powder. (F8) stability of a 20% w/w THC inhalation powder with no DSPC added; (F9) stability of a 20% w/w THC, 5% w/w DSPC inhalation powder; and (F10) stability of a 20% w/w THC, 20% w/w DSPC inhalation powder.

FIG. 5. shows the stability of 30% w/w THC inhalation powders with varying percentages of DSPC expressed as % w/w of the inhalation powder. (F18) stability of a 30% w/w THC, 5% w/w DSPC inhalation powder; (F19) stability of a 30% w/w THC, 15% w/w DSPC inhalation powder; and (F20) stability of a 30% w/w THC, 20% w/w DSPC inhalation powder.

FIG. 6. shows the stability of 20% w/w THC inhalation powders, with varying percentages of DSPC expressed as % w/w of the inhalation powder, with and without a nitrogen (N₂) atmosphere. (F8) stability of 20% w/w THC inhalation powder with no DSPC added; (F9) stability of a 20% w/w THC, 5% w/w DSPC inhalation powder; (F10) stability of a 20% w/w THC, 20% w/w DSPC inhalation powder; (F13) stability of a 20% w/w THC inhalation powder with no DSPC added and an N₂ overlay; (F14) stability of a 20% w/w THC, 5% w/w DSPC inhalation powder with an N₂ overlay; and (F15) stability of a 20% w/w THC, 20% w/w DSPC inhalation powder with an N₂ overlay.

FIG. 7. shows the stability of 26% w/w cannabidiol (CBD) inhalation powders with varying percentages of DSPC expressed as % w/w of the inhalation powder: 10% DSPC; 15% DSPC; and 20% DSPC. A 26% w/w CBD, 0% w/w DSPC inhalation powder served as a control. The data are reported as % CBD remaining after storage.

FIG. 8. shows the stability of 26% w/w CBD) inhalation powders with varying percentages of DPPC expressed as % w/w of the inhalation powder: 15% DPPC and 20% DPPC. A 26% w/w CBD, 0% w/w DPPC inhalation powder served as a control. The data are reported as % CBD remaining after storage.

FIG. 9. shows the stability of 26% w/w CBD inhalation powders with varying percentages of DSPC expressed as % w/w of the inhalation powder: 5% DSPC; 10% DSPC; 15% DSPC; and 20% DSPC. 26% w/w CBD, 0% w/w DSPC inhalation powders served as controls. The solid lines show data for the inhalation powders of FIG. 7, and the dotted lines show data for follow up inhalation powders having 0%, 5%, and 10% w/w DSPC analyzed after 24 weeks of storage. The data are reported as % CBD remaining after storage.

FIG. 10A provides exemplary cannabinoid structures. FIG. 10B provides additional exemplary structures of cannabinoids that can be synthetically derived (THC, nabilone, CBD, 7-OH-CBD, cannabidivarin (CBDV), 7-OH-CBDV, and formulas I-XVI).

FIG. 11 provides modified amino acids of compounds XVII-LI.

FIG. 12 provides fatty acid amino acids of formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH3 (i.e. methyl group), and R3 is H; or a salt or the free acid form thereof.

### DETAILED DESCRIPTION

Cannabinoids are a diverse class of compounds that interact with and activate cannabinoid receptors. There are three classes of cannabinoids: 1) endocannabinoids, which are naturally produced in the body by humans and other animals, 2) phytocannabinoids, which are produced by plants, and 3) synthetic cannabinoids, which are chemically produced cannabinoids. Synthetic cannabinoids can be identical to cannabinoids that are found in nature or can be compounds that do not exist in nature.

(−)-trans-delta-9-tetrahydrocannabinol (Δ⁹-THC) is the most extensively studied cannabinoid and has many well-established health benefits. THC is prescribed under the pharmaceutical drug name dronabinol, and is FDA approved for use as an appetite stimulant for HIV- and AIDS-related weight loss and for chemotherapy-induced nausea and vomiting. Many other medical uses of THC are being investigated, and research indicates that THC may have anti-tumor activity (Guzman M, Nat Rev Cancer. 2003. 3:745-55), anti-inflammatory effects (Gaiffal E, et al. Allergy. 2013. 68(8): 994-1000), and analgesic effects (Pharm. J. 259, 104, 1997 and in Pharm. Sci. 3, 546, 1997).

Nabilone, a synthetic cannabinoid not found in nature, is another cannabinoid that has numerous medical uses. Nabilone, which is structurally very similar to THC, has been reported to be an anti-emetic and anxiolytic, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7).

Another cannabinoid with well-documented health benefits is cannabidiol (CBD). In contrast to THC, CBD does not exert psychoactive effects. CBD is reported to have antidepressant (Zanelati T, et al. Journal of Pharmacology. 2010. 159(1):122-8;), anti-anxiety (Resstel B M, et al. Br J Pharmacol. 2009. 156(1):181-188), anti-inflammatory (Vuolo F, et al. Mediators of Inflammation. 2015. 538670), and neuroprotective effects (Campos A C, et al. Pharmacol Res. 2016. 112:119-127).

While cannabinoids have numerous health benefits, challenges to their formulation arise due to issues surrounding their stability during storage. For example, THC extracts are generally stored under refrigerated conditions and/or under inert conditions (e.g., stored under nitrogen) to improve the shelf life (chemical stability) of the THC. These storage requirements add significant expense and logistics to the storage of THC-based compositions. Moreover, while cannabinoids generally show increased stability when diluted into liquid formulations, a need for stabilized cannabinoids within solid dosage forms remains.

Currently, cannabinoid medicines are delivered as oral capsules, oral solutions, or buccal sprays. These products are characterized by long duration to onset, low bioavailability, and high variability. Thus, there is an unmet medical need in cannabinoid-based therapies for dosage forms that provide precise delivery and improved bioavailability.

Phosphatidylcholines (PC) have a number of uses in the formulation of administrable compositions. For example, 2-distearoyl-sn-glycero-3-phosphocholine (DSPC) and 1,2-dipalmitoyl-sn-glycero-3-phosphocholine (DPPC) are approved pharmaceutical excipients that are used as surfactants, microcarriers, and bulking agents. When used as a surfactant, DSPC and DPPC have been shown to stabilize oil-in-water emulsions (see, e.g., U.S. Pat. Nos. 6,383,513 and 10,328,216). DSPC and DPPC are also commonly used to prepare liposomes, which may be dried into powders and administered by inhalation.

The current disclosure provides that the use of PC, such as DSPC and DPPC, can be used to directly stabilize cannabinoids within a solid dosage form, such as a dry powder for inhalation or tablet or capsule formation. By “directly stabilize” it is meant that cannabinoids within a PC-containing dry powder will degrade by no more than 15%, 10%, 5%, 4%, 3%, 2%, or 1% upon storage of the dry powder under ambient conditions for at least 1 week, at least 2 weeks, at least 4 weeks, at least 6 weeks, at least 10 weeks, at least 15 weeks, at least 20 weeks, at least 32 weeks, at least 36 weeks, at least 48 weeks, at least 60 weeks, or at least 63 weeks. Cannabinoid content and degradation can be measured using high performance liquid chromatography (HPLC). Degradation can also be measured as a loss of function. Particular embodiments include a dry powder including 5-30% PC and 5-50% cannabinoid or a dry powder including 5-20% PC and 10-30% cannabinoid. Particular embodiments include a dry powder including 5-30% DSPC and/or DPPC and 5-50% cannabinoid or a dry powder including 5-20% DSPC and/or DPPC and 10-30% cannabinoid. Particular embodiments include a dry powder including 5-30% PC and 5-50% THC and/or CBD or a dry powder including 5-20% PC and 10-30% THC and/or CBD. Particular embodiments include a dry powder including 5-30% DSPC and/or DPPC and 5-50% THC and/or CBD or a dry powder including 5-20% DSPC and/or DPPC and 10-30% THC and/or CBD.

Aspects of the current disclosure are now described with additional detail and options as follows: (i) Cannabinoids; (ii) Phospholipids; (iii) Dry Powders; (iv) Dry Powders for Inhalation; (v) Dry Powders for Nasal Administration; (vi) Dry Powders for Oral Compositions; (vii) Methods of Use; (viii) Exemplary Embodiments; (ix) Experimental Examples; and (x) Closing Paragraphs. These headings do not limit the interpretation of the disclosure and are provided for organizational purposes only.

(i) Cannabinoids. Cannabinoids are a group of cyclic molecules from cannabis plants that activate cannabinoid receptors (i.e., CB1 and CB2) in cells. There are at least 85 different cannabinoids that can be isolated from cannabis. The most notable cannabinoids are THC and CBD. Additional examples include cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCVA). Extracts of the cannabis plant similarly include flavonoid compounds, terpenes, terpenoid, and synthetic, semisynthetic or highly purified versions of any such constituent.

In particular embodiments, synthetic cannabinoids include natural cannabinoids that are synthesized chemically and also their analogs and derivatives. Derivatives of natural cannabinoids can include metabolites of cannabinoids which are disclosed in WO2015/198078. For example, the metabolite of CBD includes 7-OH-CBD and the metabolite of CBDV includes 7-OH-CBDV.

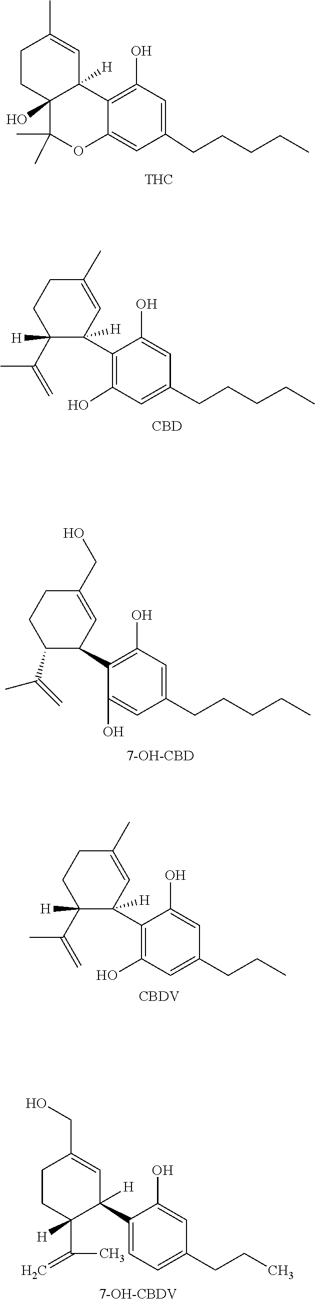

Other examples of synthetic cannabinoids include 3-carbamoyl-2-pyridone, and its derivatives and/or analogs disclosed in US2008/0103139; pyrimidine derivatives and/or analogs disclosed in US2006/0293354; carenadiol and its derivatives and/or analogs thereof disclosed in U.S. Pat. No. 4,758,597; cannabinoid carboxylic acids and their derivatives and/or analogs disclosed in WO2013/045115; pyrido[3,2-E][1,2,4]triazolo[4,3-C]pyrimidine and its derivatives and/or analogs disclosed in WO2008/118414; tetrahydro-pyrazolo[3,4-C] pyridine and its derivatives and/or analogs disclosed in WO2007/112399; bicyclo[3.1.1]heptan-2-one cannabinoid and its derivatives and/or analogs disclosed in WO2006/043260; resorcinol and its derivatives and/or analogs disclosed in WO2005/0123051; dexanabinol compounds and their derivatives and/or analogs disclosed in WO2004/050011; cannabimimetic lipid amide compounds and their derivatives and/or analogs disclosed in WO2000/032200; nabilone and its derivatives and/or analogs disclosed in US2010/0168066; 2-oxoquinolone compounds and their derivatives and/or analogs disclosed in US2003/0191069; and 3,4-diaryl-4,5-dihydro-(h)-pyrazole-1-carboxamide and its derivatives and/or analogs disclosed in US2011/0137040.

In particular embodiments, 3-carbamoyl-2-pyridone and its derivatives and/or analogs include methyl 3-methyl-2-{[2-oxo-1-(2-oxo-ethyl)-1,2,5,6,7,8,9,10-octahydro-cycloocta[b]pyridine-3-carbonyl]-amino}-butyrate; dimethyl 2-[(1-cyclohexylmethyl-5,6-dimethyl-2-oxo-1,2-dihydropyridine-3-carbonyl)-amino]-succinate; and methyl 2-{[1-(3-methoxycarbonyamino-propyl)-2-oxo-1,2,5,6,7,8,9,10-octahydro-cycloocta[b]pyridine-3-carbonyl]-amino}-2-methyl-propionate.

In particular embodiments, pyrimidine derivatives and/or analogs include a compound having Formula (I) (2-((2,4-dichlorophenyl)amino)-N-((tetrahydro-2H-pyran-4-yl)methyl)-4-(trifluoromethyl)pyrimidine-5-carboxamide),

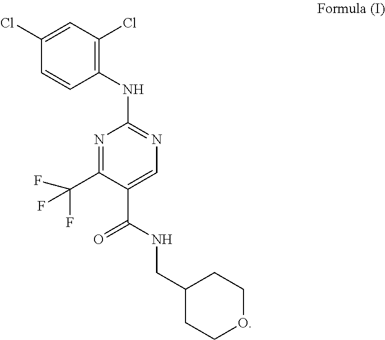 [SMILES: O=C(NCC1CCOCC1)c1cnc(Nc2ccc(Cl)cc2Cl)nc1C(F)(F)F]

Other pyrimidine derivatives and/or analogs include 2-(3-Chlorophenylamino)-4-trifluoromethylpyrimidine-5-carboxylic acid cyclohexylmethyl-amide; 2-Phenylamino-4-trifluoromethylpyrimidine-5-carboxylic acid cyclohexylmethyl-amide; 1-[2-(2,3-Dichlorophenylamino)-4-trifluoromethylpyrimidin-5-yl]-1-morphol-in-4-yl-methanone; 1-[2-(2,4-Dichlorophenylamino)-4-trifluoromethylpyrimidin-5-yl]-1-morphol-in-4-yl-methanone; and 2-(3-Chlorophenylamino)-4-trifluoromethylpyrimidin-5-carboxylic acid cyclopentylamide.

In particular embodiments, carenadiol and its derivatives and/or analogs include compounds having Formula (II),

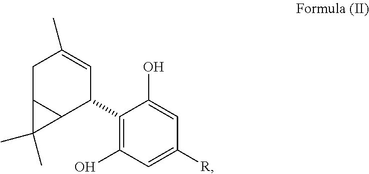

wherein R is a lower alkyl having 1 to 9 carbon atoms including isomeric forms such as i-butyl, n-butyl, and t-butyl. In particular embodiments, R is C₅H₁₁ or 1,1-dimethylheptyl.

In particular embodiments, cannabinoid carboxylic acids and their derivatives and/or analogs include compounds having Formula (III), (IV), (V), or (VI),

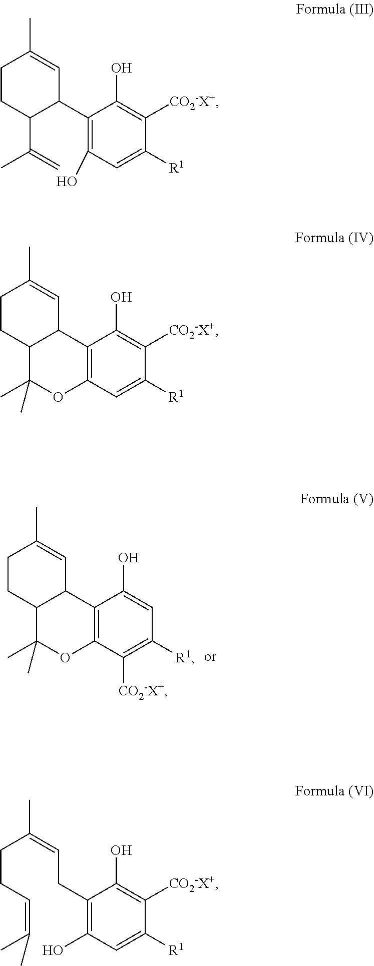

wherein:
R¹ is a straight-chain, branched or cyclic hydrocarbon residue with one C atom to 12 C atoms; and
X⁺ is NH₄⁺, mono-, di- or trivalent metal ions; or primary, secondary, tertiary or quaternary organic ammonium ions with up to 48 C atoms, which may bear still further functional groups.

Examples of multivalent ammonium ions include N,N-dicyclo-hexylamine-H⁺ and N,N-dicyclohexyl-N-ethylamine-H⁺. X⁺ can also be the hydrogen cation of a pharmaceutical active substance with at least one basic nitrogen atom, such as for example morphine, methadone (or an enantiomer thereof) or hydromorphone.

In particular embodiments, pyrido[3,2-E][1,2,4]triazolo[4,3-C]pyrimidine and its derivatives and/or analogs include 5-tert-butyl-8-(2-chlorophenyl)-9-(4-chlorophenyl)pyrido[3,2-e][1,2,4]triazolo[4,3-c]pyrimidin-3(2H)-one; 8-(4-bromo-2-chlorophenyl)-5-tert-butyl-9-(4-chlorophenyl)pyrido[3,2-e][1,2,4]triazolo[4,3-c]pyrimidin-3(2H)-one; 5-tert-butyl-9-(4-chlorophenyl)-8-(2-methylphenyl)pyrido[3,2-e][1,2,4]triazolo[4,3-c]pyrimidin-3(2H)-one; 9-(4-bromophenyl)-5-tert-butyl-8-(2-chlorophenyl)pyrido[3,2-e][1,2,4]triazolo[4,3-c]pyrimidin-3(2H)-one; and 5-tert-butyl-8-(2-chlorophenyl)-9-(4-chlorophenyl)pyrido[3,2-e][1,2,4]triazolo[4,3-c]pyrimidine.

In particular embodiments, tetrahydro-pyrazolo[3,4-C] pyridine and its analogs and/or derivatives include compounds having Formula (VII), (VIII), (IX), (X), or (XI),

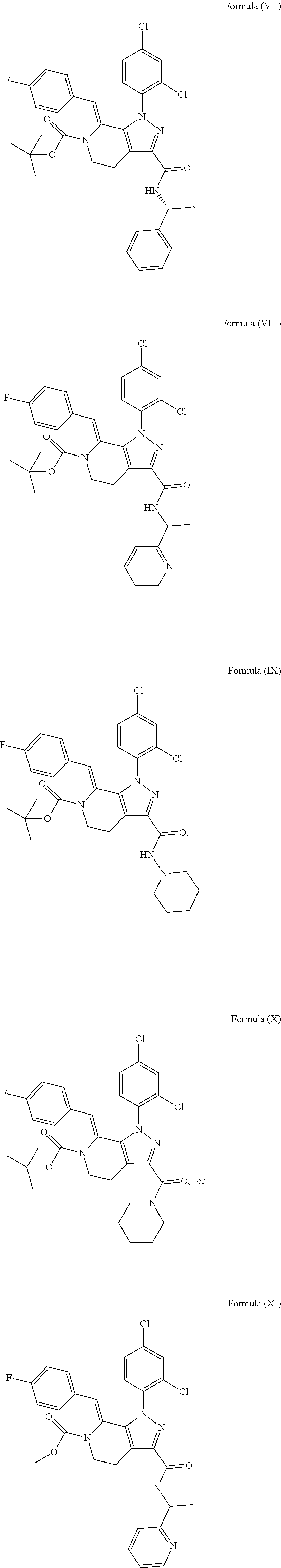 [SMILES: C[C@@H](NC(=O)c1nn(-c2ccc(Cl)cc2Cl)c2c1CCN(C(=O)OC(C)(C)C)/C2=C\c1ccc(F)cc1)c1ccccc1 ; CC(NC(=O)c1nn(-c2ccc(Cl)cc2Cl)c2c1CCN(C(=O)OC(C)(C)C)/C2=C\c1ccc(F)cc1)c1ccccn1 ; CC(C)(C)OC(=O)N1CCc2c(C(=O)NN3CCCCC3)nn(-c3ccc(Cl)cc3Cl)c2/C1=C/c1ccc(F)cc1 ; CC(C)(C)OC(=O)N1CCc2c(C(=O)N3CCCCC3)nn(-c3ccc(Cl)cc3Cl)c2/C1=C/c1ccc(F)cc1 ; COC(=O)N1CCc2c(C(=O)NC(C)c3ccccn3)nn(-c3ccc(Cl)cc3Cl)c2/C1=C/c1ccc(F)cc1]

In particular embodiments, bicyclo[3.1.1]heptan-2-one cannabinoids and their derivatives and/or analogs include compounds having Formula (XII),

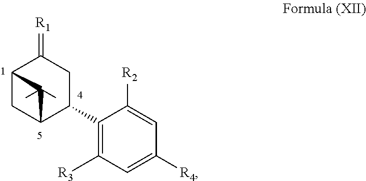

having a specific stereochemistry wherein C-4 is S, the protons at C-1 and C-5 are cis in relation to one another and the protons at C-4 and C-5 are trans; and wherein:
R₁ is (a) O or S; (b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆alkyl-N(R″)₂ wherein at each occurrence R″ is independently selected from the group consisting of hydrogen, C(O)R′″, C(O)N(R′)₂, C(S)R′″, saturated or unsaturated, linear or branched C₁-C₆ alkyl, C₁-C₆ alkyl-OR′″, and C₁-C₆ alkyl-N(R′)₂, wherein at each occurrence R′″ is independently selected from the group consisting of hydrogen or saturated or unsaturated, linear, branched or cyclic C₁-C₁₂ alkyl; or (c) NR″ or N—OR″ wherein R″ is as previously defined;
R₂ and R₃ are each independently (a) —R″, —OR″, —N(R″)₂, —SR″, —S(O)(O)NR″, wherein at each occurrence R″ is as previously defined; (b) —S(O)R^{b}, —S(O)(O)R^{b} wherein R^{b} is selected from the group consisting of hydrogen, saturated or unsaturated, linear or branched C₁-C₆ alkyl, C₁-C₆ alkyl-OR″, and C₁-C₆ alkyl-N(R″)₂, wherein R″ is as previously defined; or (c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is (a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined; (b) a saturated or unsaturated, linear, branched or cyclic C₁-C₁₂ alkyl-R wherein R is as previously defined; (c) an aromatic ring which can be further substituted at any position by R wherein R is as previously defined; or (d) a saturated or unsaturated, linear, branched or cyclic C₁-C₁₂ alkyl optionally terminated by an aromatic ring which can be further substituted as defined in (c).

In particular embodiments, resorcinol and its derivatives and/or analogs include compounds having Formula (XIII),

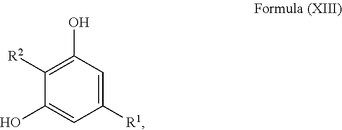

wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms.

In particular embodiments, resorcinol and its derivatives and/or analogs include compounds having Formula (XIII), wherein R¹ and R² are as follows:
R¹ is a straight alkyl chain of 5 to 8 carbon atoms, optionally substituted with one methyl group; and
R² is selected from geranyl optionally substituted with one —OH, and farnesyl optionally substituted with one —OH.

In particular embodiments, resorcinol and its derivatives and/or analogs include compounds having Formula (XIII), wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms; with the proviso that when R¹ is isononyl, R² is not geranyl.

In particular embodiments, resorcinol and its derivatives and/or analogs include compounds having Formula (XIII), wherein R¹ is (a) a straight or branched alkyl of 7 to 12 carbon atoms; (b) a group —O—R³, where R³ is a straight or branched alkyl of 5 to 9 carbon atoms, or a straight or branched alkyl substituted at the terminal carbon atom by a phenyl group; or (c) a group —(CH₂)ₙ—O-alkyl, where n is an integer from 1 to 7 and the alkyl group contains 1 to 5 carbon atoms.

In particular embodiments, resorcinol and its derivatives and/or analogs include compounds of Formula (XIII), wherein R² is a non-cyclic terpenoid carbon chain such as geranyl, farnesyl, and related non-cyclic terpenes and their isomers as well as other non-cyclic paraffinic or olefinic carbon chains.

In particular embodiments, resorcinol and its derivatives and/or analogs include compounds of Formula (XIII), wherein R¹ is dimethylheptyl and R² is geranyl.

In particular embodiments, dexanabinol compounds and their derivatives and/or analogs include high enantiomeric purity compounds having Formula (XIV),

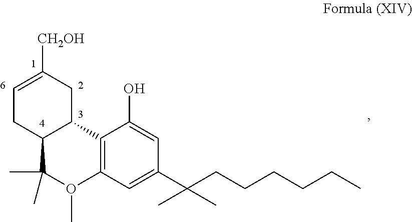

and having the (3S, 4S) configuration and being in enantiomeric excess of at least 99.90% over the (3R,4R) enantiomer.

In particular embodiments, cannabimimetic lipid amide compounds and their derivatives and/or analogs include compounds having Formula (XV),

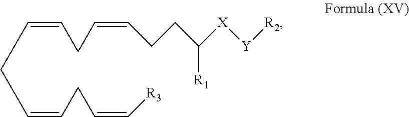

wherein:
X is one of the group consisting of C═O and NH, and Y is the other of that group. Expressed another way, X may be C═O and Y may be NH, or Y may be C═O and X may be NH, but both X and Y may not be the same group.
R₁ is H or an alkyl group. In particular embodiments, R₁ is H, CH₃, or (CH₃)₂;
R₂ is an alkyl, a substituted alkyl, an alkenyl or an alkynyl group. In particular embodiments,
R₂ is CH(R) CH₂Z, CH₂CH(R)Z, or CH(R)(CH₂)ₙCH₂Z; R being H, CH, CH₃, CHCH, CH₂CF₃, or (CH₃)₂; Z being H, halogen, N₃, NCS, or OH; and n being selected from the group consisting of 0, 1 and 2.
R₃ is an alkyl, a substituted alkyl, an aryl, an alkylaryl, an O-alkyl, an O-alkylaryl, a cyclic and a heterocyclic group. O-alkyl and O-alkylaryl refer to groups in which an oxygen atom is interposed between carbon atoms on the anandamide portion and substituent group. Examples of such R₃ groups include cyclohexyl, cyclopentyl, alkylcyclohexyl, alkylcyclopentyl, piperidinyl, morpholinyl and pyridinyl. In particular embodiments, R₃ is n-C₅H₁₀Z′, n-C₆H₁₂Z′, n-C₇H₁₄Z′, or 1′, 1′—C(CH₃)₂(CH₂)₅ CH₂Z′; Z being H, halogens, CN, N₃, NCS, or OH.

In particular embodiments, cannabimimetic lipid amide compounds and their derivatives and/or analogs include compounds having Formula (XVI),

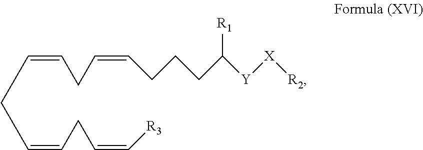

wherein:
Y is one of the group consisting of C═O and NH and X is the other of that group.
R₁ is H or an alkyl group. In particular embodiments, R₁ is H, CH₃, or (CH₃)₂.
R₂ is an alkyl, a substituted alkyl, an alkenyl, an alkynyl, an O-alkyl, a cyclic, a polycyclic, or a heterocyclic group. In particular embodiments, R₂ is

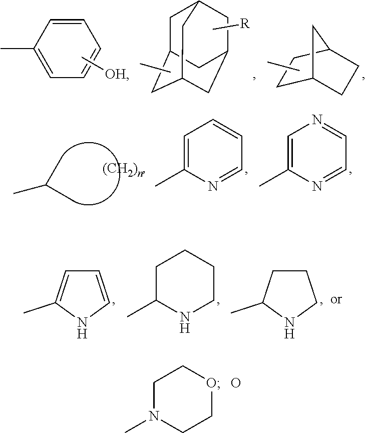

CH═CH₂, CH═C(CH₃)₂, C≡CH, CH₂OCH₃, CH(R)(CH₂)ₙCH₂Z, or CH₂CH(R)(CH₂)ₙZ; R being H, CH₃ or (CH₃)₂; Z being H, halogens, N₃, NCS, OH, or OAc; and n 0, 1, or 2; and
R₃ is an alkyl, a substituted alkyl, an aryl, an alkylaryl, an O-alkyl, an O-alkylaryl, a cyclic, or a heterocyclic group. In particular embodiments R₃ includes cyclohexyl, cyclopentyl, alkylcyclohexyl, alkylcyclopentyl, piperidinyl, morpholinyl and pyridinyl. In particular embodiments, R₃ is n-C₅H₁₀Z′, n-C₆H₁₂Z′, n-C₇H₁₄Z′, or 1′,1′-C(CH₃)₂CH₂)₅ CH₂Z′; Z′ being H, halogen, CN, N₃, NCS, or OH.

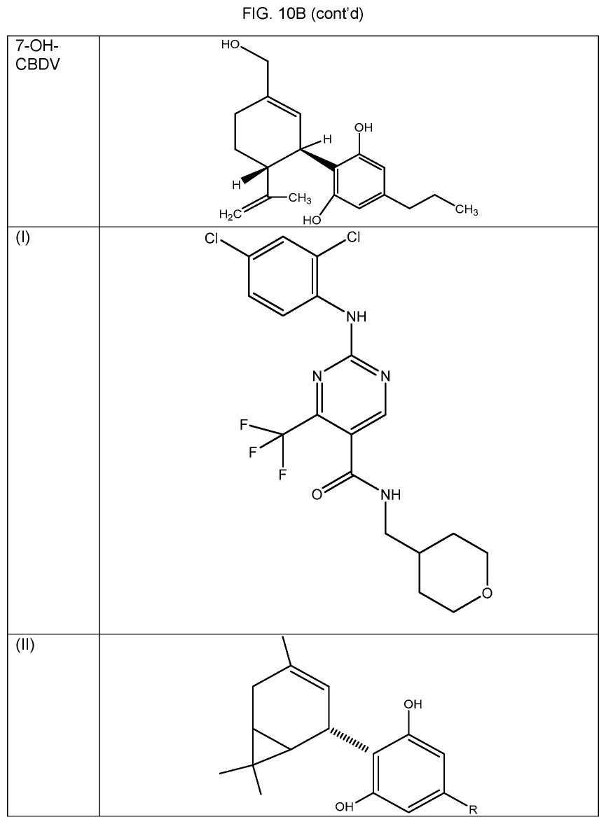

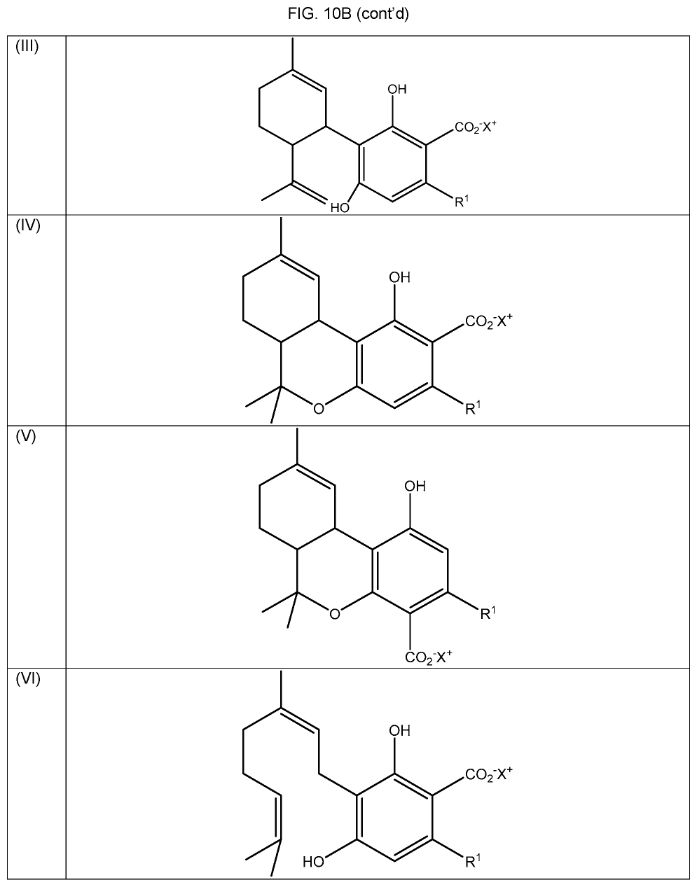

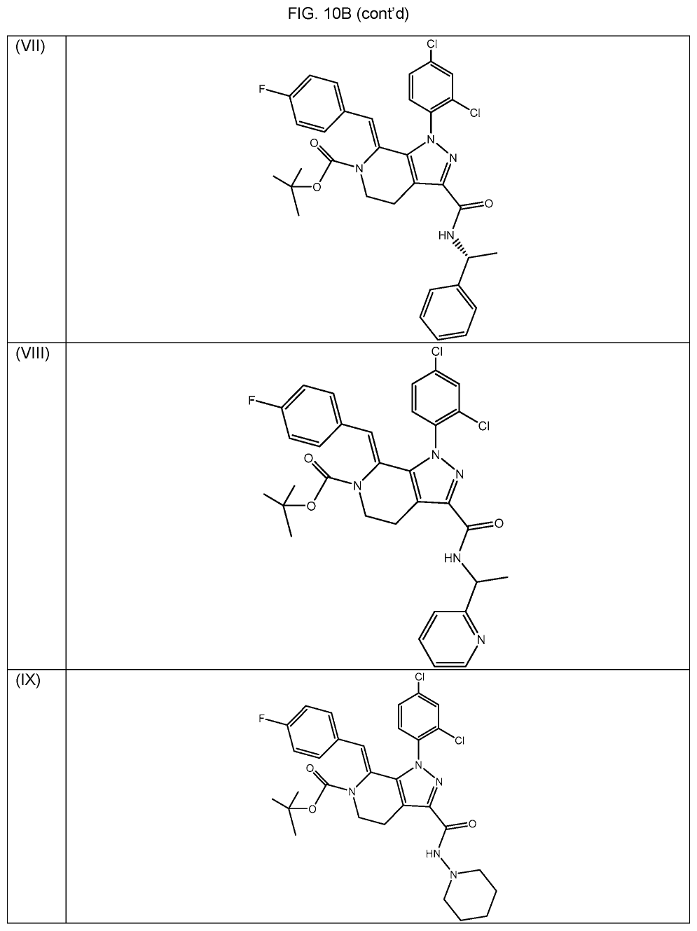

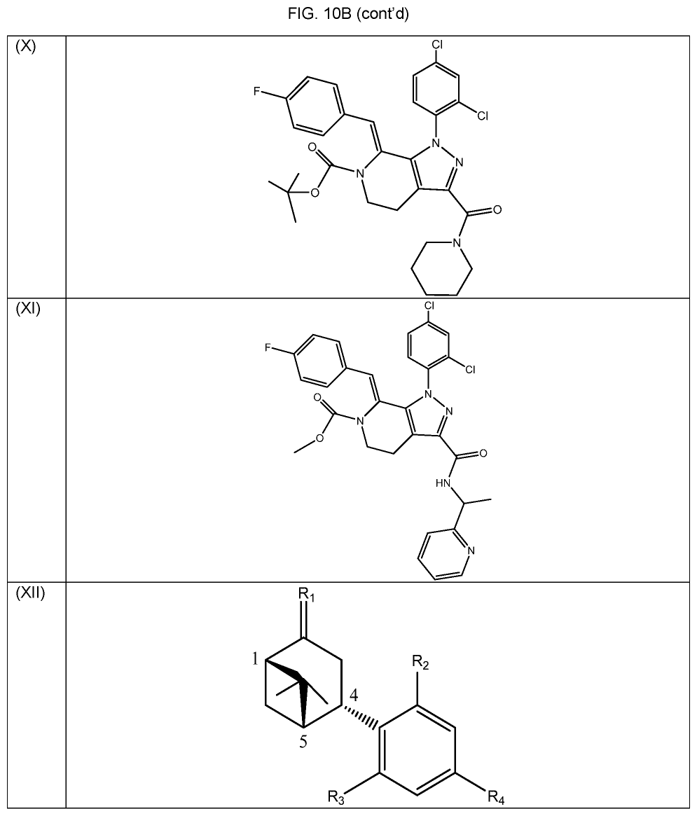

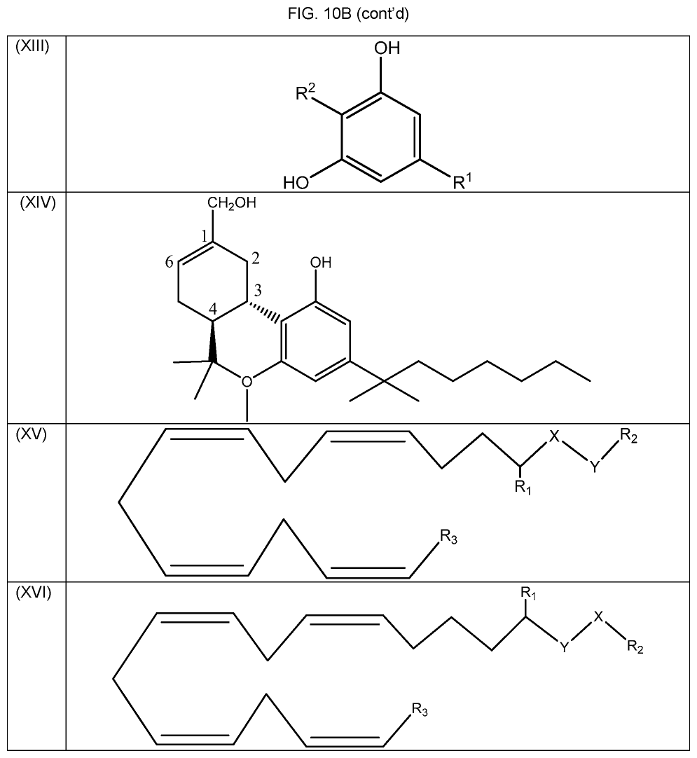

In particular embodiments, nabilone and its derivatives and/or analogs include compounds having Formula (LII):

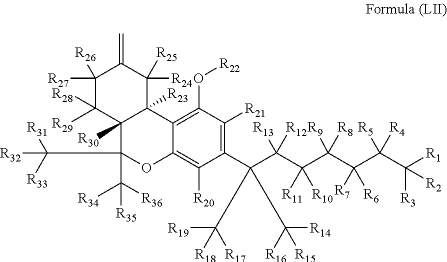

wherein:
   R₁-R₃₆ are independently selected from the group consisting of hydrogen and deuterium. Nabilone derivatives and/or analogs can refer to compounds wherein at least one of R₁-R₃₆ includes deuterium. For the chemical structure of nabilone, see FIG. 10B.

Cannabinoids can be medicinal compounds and/or can be provided in combination with nutritional supplements.

(ii) Phospholipids. Phospholipids carry one or two, more usually two C8-32 alkyl groups bound to a polar phosphorylated alcohol headgroup. The alkyl groups may be straight or branched chain, saturated or unsaturated, and may be optionally substituted, for example, by one or more hydroxyl groups.

Phosphatidylcholines (PC) are a class of phospholipids that incorporate choline as a headgroup. PC is a component of cell membrane bilayers and the main phospholipid circulating in plasma. PC is highly absorbable and supplies choline which is needed to facilitate movement of fats and oils across the cell membrane, and to maintain the cell membrane.

PC typically possess a hydrophilic head group and a hydrophobic tail group, which is saturated or unsaturated. Hydrophobic tail groups are generally saturated fatty acid groups or derivatives thereof. The term “fatty acid” refers to linear or branched, saturated or unsaturated carboxylic acids or derivatives thereof including a carbon chain of C₇₋₂₄. Typically, saturated fatty acids comprise a carbon chain of C₁₀₋₂₄, or C₁₀₋₂₀. Representative fatty acids include, for example, oleic acid, lauric acid, capric acid (n-decanoic acid), myristic acid, palmitic acid, stearic acid, linoleic acid, and linolenic acid.

Typically, PC or derivatives there have the general structure:

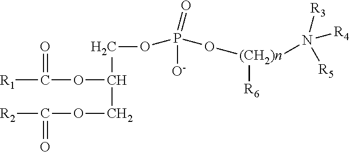

wherein R₁ and R₂ are, independently, hydrocarbon-based, substantially hydrophobic “tail” groups;
R₃, R₄, R₅, R₆ are independently H or alkyl; and
n is an integer from 1-4. As indicated, choline, together with the phosphate moiety, forms a substantially hydrophilic “head”.
PC compounds can also be designated by the general structure:

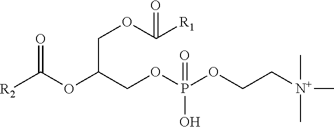

wherein R₁ and R₂ are each independently an aliphatic group, saturated or unsaturated, having from 9 to 21 carbon atoms, suitably 13 to 21 carbon atoms, and preferably 13 to 17 carbon atoms.

Examples of PCs include 1,2-dipalmitoyl-sn-glycero-3-phosphocholine (dipalmitoylphosphatidylcholine; DPPC), 1,2-distearoyl-sn-glycero-3-phosphocholine (distearoylphosphatidylcholine; DSPC), 1-oleoyl-2-stearoyl-sn-glycero-3-phosphocholine (OSPC), 1,2-dilauroyl-sn-glycero-3-phosphocholine (DLPC), 1,2-dimyristoyl-sn-glycero-3-phosphocholine (dimyristoylphosphatidyl choline; DMPC), 1,2-dioleoyl-sn-glycero-3-phosphocholine (DOPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), 1-stearoyl-2-myristoyl-sn-glycero-3-phosphocholine (SMPC), 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), and 1-oleoyl-2-palmitoyl-sn-glycero-3-phosphocholine (OPPC).

An exemplary DSPC analog includes

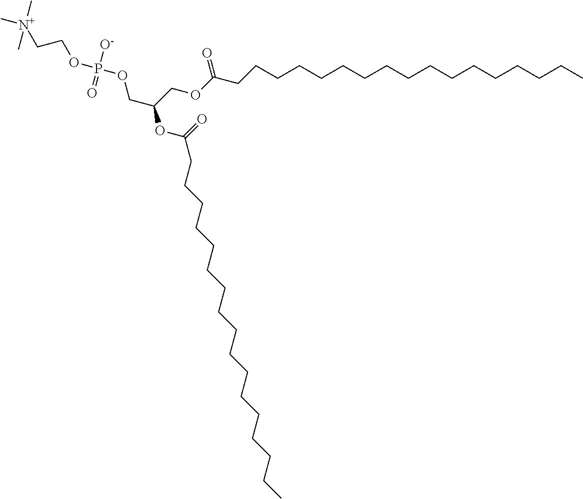 [SMILES: CCCCCCCCCCCCCCCCCC(=O)OC[C@H](COP(=O)([O-])OCC[N+](C)(C)C)OC(=O)CCCCCCCCCCCCCCCCC]

as described in US20190175727.

Particular embodiments utilize a phosphatidylcholine lipid comprising fatty acid chains of C₃₋₃₂, C₇₋₂₄, C₃₋₂₂, or C₁₀₋₂₀. Specific examples of such lipids include DSPC, DPPC, dioleoylphosphatidylcholine (DOPC), palmitoyloleoylphosphatidylcholine (POPC).

An exemplary DPPC analog includes

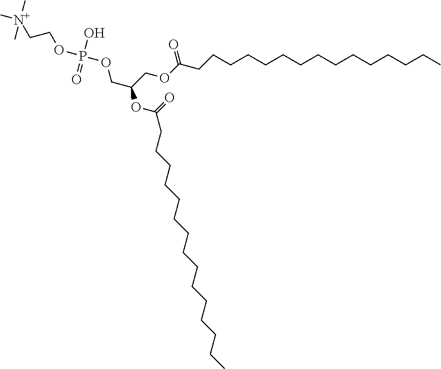 [SMILES: CCCCCCCCCCCCCCCC(=O)OC[C@H](COP(=O)(O)OCC[N+](C)(C)C)OC(=O)CCCCCCCCCCCCCCC]

as described in U.S. Pat. No. 8,658,205.

1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine are additional examples of PCs.

In particular embodiments, PC can be isolated from natural sources (e.g., soy, egg, cotton seed, sunflower seed), in which a variety of PC derivatives with a variety of head groups of varying chain length and degree of saturation may be present. For example, egg PC contains 28-38% palmitic acid, 9-18% stearic acid, 25-37% oleic acid, 12-17% linoleic acid, 0.5% linolenic acid and 1-7% arachidonic acid. Soy PC contains mainly stearic and palmitic acids.

In particular embodiments, the PC is described as 13% C16:0 (palmitic), 4% C18:0 (stearic), 10% C18:1 (oleic), 64% C 18:2 (linoleic), and 6% 18:3 (linolenic) with other fatty acids being minor contributors. Particular embodiments include linoleic acid at a percentage of up to 70% of the total fatty acids.

In other embodiments, essentially pure synthetic PC which are commercially available (e.g., Sigma-Aldrich) or which may be synthesized by known techniques can be used.

PCs may also be provided as a mixture of natural and synthetic PC.

Other suitable phospholipids include phosphatidyl compounds with head groups derived from alcohols other than choline, such as, for example, ethanolamine, serine, glycerol, and inositol. Examples of phospholipids including phosphatidyl compounds with head groups derived from alcohols other than choline include dipalmitoyl phosphatidyl ethanolamine (DPPE); sphingomyelin (Sphg); dipalmitoyl phosphatidyl serine (DPPS); dipalmitoyl phosphatidyl glycerol (DPPG); and dipalmitoyl phosphatidyl inositol (DPPI).

(iii) Dry Powders. Particular embodiments described herein include a cannabinoid directly stabilized by a PC within a dry powder. As used herein a dry powder refers to a fine particulate composition that is not suspended or dissolved in a propellant, carrier, or other liquid. It does not imply a complete absence of all water molecules. Instead, the moisture content of a dry powder can be less than 10%, less than 8%, less than 6%, less than 4%, less than 2% or less than 1% by weight of the powder.

Dry powders can be characterized according to a number of parameters including, for example, particle size, bulk density, percent of non-monomeric forms, and rugosity.

Particle sizes can correspond to actual physical diameter or aerodynamic diameter. The average particle size of the powder may also be measured as mass mean diameter (MMD) by conventional techniques.

In particular embodiments, the particle size of a dry powder is assessed according to particle size distribution within the powder. The particle-size distribution (PSD) of a powder is a list of values or a mathematical function that defines the relative amount of particles present according to size. In particular embodiments, powders are polydispersed (i.e., having a range of particle sizes). In particular embodiments, the term “particle size distribution” refers to the size distribution of a particle system and represents the number of particles that fall into each of the various size ranges, given as a percentage of the total solids of all sizes in the sample of interest.

A “particle size distribution D90 value” is the numerical value, expressed in microns (μm), at which 90 percent of the particles have particle sizes which are less than or equal to that value. A “particle size distribution D50 value” is the numerical value, expressed in microns, at which 50 percent of the particles have particle sizes which are less than or equal to that value.

Particles sizes in some embodiments are in the range of 0.5-50 μm, 0.5 μm-20.0 μm, or 0.5 μm-8.0 μm. According to some embodiments, the average particle size is below 10 μm. In other embodiments, the average particle size is below 9, 8, or 7 μm. In additional embodiments, the average particle size is between 1 to 10 μm, 2 to 9 μm, 3 to 8 μm, or 4 to 8 μm.

Dry powders can also be characterized by their densities. According to some embodiments, the dry powder includes particles having a bulk density from 0.1 to 10 grams per cubic centimeter. According to certain embodiments, the dry powder includes particles having a bulk density from 0.1 to 2 grams per cubic centimeter. According to additional embodiments, the dry powder includes particles having a bulk density from 0.15 to 1.5 grams per cubic centimeter.

An additional measure for characterizing dry powders includes the non-monomeric forms percent (NMF %), which implies the aggregation level of the powder after reconstitution as measured by size exclusion chromatography-high performance liquid chromatography (SEC-HPLC). According to some embodiments, the NMF % of the reconstituted powder is less than 8%, less than 7%, or less than 6%.

In certain embodiments, particles of dry powders have a rugosity of less than 2.

Dry powders can include pharmaceutically-acceptable salts and excipients. Exemplary salts include those prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically-acceptable cations including sodium, potassium, calcium, aluminum, lithium, and ammonium cations may be used.

Exemplary pharmaceutically-acceptable excipients that are used in dry powders include fumaryl diketopiperazine (2,5-diketo-3,6-bis(N-fumaryl-4-aminobutyl)piperazine; FDKP), sodium N-[8-(2-hydroxybenzoyl) amino] caprylate (SNAC), amino acids (e.g., leucine, isoleucine), mannitol, lactose, polyvinylpyrrolidones, derivatized celluloses (e.g., hydroxymethylcellulose, hydroxyethylcellulose, and hydroxypropylmethylcellulose), Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, pectin, flavoring agents, taste-masking agents, sweeteners, antioxidants, antistatic agents, surfactants other than phospholipids used in stabilization of cannabinoids as described herein (e.g., polysorbates such as “TWEEN 20” and “TWEEN 80”), sorbitan esters, lipids, fatty acids and fatty esters, steroids (e.g., cholesterol), and chelating agents (e.g., EDTA, zinc and other such suitable cations). Other pharmaceutical excipients and/or additives suitable for use in the dry powder compositions are listed, for example, in “Remington: The Science & Practice of Pharmacy”, 22^{th} ed., Allen et al., (1995), and in the “Physician's Desk Reference”.

In certain embodiments, excipients are selected to reduce or prevent aggregation of particles, and as a result, improve the dispersibility of the powder.

Dry powders can be prepared by any known method in the art (e.g., spray drying, micronization, and the like). According to some embodiments, a liquid formulation is spray-dried to produce a dry powder. Spray drying of the formulations is carried out, for example, as described generally in the “Spray Drying Handbook”, 5th ed., K. Masters, John Wiley & Sons, Inc., NY, N.Y. (1991), and in International application WO 97/41833.

In one example, utilizing the spray-drying approach, cannabinoids are first mixed into a solvent. In particular embodiments, the solvent includes ethanol, methanol, acetone, tetrahydrofuran, or ethyl acetate. In particular embodiments, the solvent includes ethanol and water, methanol and water, acetone and water, or tetrahydrofuran and water. The pre-spray dried solutions will generally contain cannabinoids at a concentration from 0.01% (weight/volume) to 80% (weight/volume), usually from 0.1% to 60% (weight/volume) of the solvent. In particular embodiments, the w/w ratio of cannabinoid to solvent is 1:100. The solvent including cannabinoids can then be mixed with a phospholipid (e.g., DSPC or DPPC) to form a cannabinoid/phospholipid solution. In particular embodiments, the cannabinoid and the phospholipid may be mixed at a w/w ratio including 1:1, 1:2, 1:3, 1:4, 1:5, 1:6, 5:1, 4:1, 3:1, and 2:1. Excipients can be added to the cannabinoid/phospholipid solution. For example, FKDP can first be added to a solvent (e.g., ethanol) and stirred until a uniform suspension is obtained. In particular embodiments, the solvent includes ethanol, methanol, acetone, tetrahydrofuran, or ethyl acetate. In particular embodiments, the solvent includes ethanol and water, methanol and water, acetone and water, or tetrahydrofuran and water. In particular embodiments, the solvent in which the excipient is suspended is the same solvent in which the cannabinoids are dissolved. The excipient suspension can then be added to the cannabinoid/phospholipid solution and mixed. In particular embodiments, water can be added to the cannabinoid/phospholipid/excipient suspension and mixed. The resulting suspension can then be spray dried to form a dry powder. A suspension refers to a mixture of fine, non-settling particles of a solid within a liquid phase.

The solutions are then spray dried in a conventional spray drier, such as those available from commercial suppliers such as Niro A/S (Denmark), Buchi (Switzerland), Eurotherm (US) and the like, resulting in a dispersible, dry powder. Optimal conditions for spray drying the solutions will vary depending upon the particular formulation components and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition of the cannabinoids in the sprayed material. Such temperatures are typically determined experimentally, although generally, the inlet temperature will range from 50° C. to 200° C., while the outlet temperature will range from 30° C. to 150° C.

In particular embodiments, dry powder is prepared by spray-drying a suspension, as described in U.S. Pat. No. 5,976,574. In this method, cannabinoids are mixed in an organic solvent, e.g., methanol, ethanol, isopropanol, acetone, heptane, hexane, chloroform, or ether to form a suspension. The suspension is then spray-dried to form particles. Exemplary solvents, for both of the above spray-drying methods include alcohols, ethers, ketones, hydrocarbons, polar aprotic solvents, and mixtures thereof.

In additional embodiments, dry powders can be formed using lyophilization, vacuum drying, spray freeze drying, super critical fluid processing, air drying, or other forms of evaporative drying. Dry powders may also be manufactured by additional processes well known in the art, e.g. by means of conventional mixing, dissolving, granulating, grinding, pulverizing, dragee-making, levigating or lyophilizing processes. For example, dry powders can be prepared by agglomerating the powder components, sieving the materials to obtain agglomerates, spheronizing to provide a more spherical agglomerate, and sizing to obtain a uniformly-sized product, as described, e.g., in WO 95/09616.

Once formed, the dry powder compositions are preferably maintained under dry (i.e., relatively low humidity) conditions during manufacture, processing, and storage. Relatively low humidity can include a humidity below 30%, below 25%, below 20%, below 15%, below 10%, below 5%, or lower. In particular embodiments, the dry powder compositions are maintained at uncontrolled ambient conditions. Uncontrolled ambient conditions refers to conditions of being indoors at room temperature (20° C.-25° C.), with normal humidity of 30-60% and no attempt to control either temperature or humidity fluctuations. These conditions are selected because they are representative of patient home storage conditions. In particular embodiments, the dry powder compositions are maintained at a temperature of 25° C. and 60% relative humidity. In particular embodiments, the dry powder compositions are maintained at a temperature of 23° C., 24° C., 25° C., or 26° C., and at 58%, 59%, 60%, 61%, or 62% relative humidity. In particular embodiments, the dry powder compositions are stored in tightly closed amber glass vials at a temperature of 23° C., 24° C., 25° C., or 26° C., and at 58%, 59%, 60%, 61%, or 62% relative humidity. In particular embodiments, the dry powder compositions are stored in tightly closed amber glass vials at uncontrolled ambient conditions or at a temperature of 25° C. and 60% relative humidity.

In particular embodiments, the dry powders include cannabinoids at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders include THC at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders include THC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders include CBD at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders include CBD at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders include PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 0.5-5% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 5-10% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 10-20% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 20-30% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 30-40% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 40-50% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders include cannabinoids at 25% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

The remaining percentage of the dry powders can include excipients.

Dry powders described herein can also be characterized according to the ratio of cannabinoids to PC within a dry powder. This cannabinoid:PC ratio can include, for example, 1:1, 1:2, 1:3, 1:4, 1:5, 1:6, 5:1, 4:1, 3:1, or 2:1.

(iv) Dry Powders for Inhalation. Particular embodiments described herein include a cannabinoid stabilized by a PC within a dry powder for inhalation. Generally, the dry powders for inhalation are designed to be respirable powders that are suitable for pulmonary delivery. Such powders are capable of being (i) readily dispersed by an inhalation device and (ii) inhaled by a subject so that at least a relevant portion of the particles reach the lungs to permit penetration into the alveoli. In particular embodiments, an active ingredient in an inhalation dry powder (e.g., a cannabinoid) is rapidly available to a subject inhaling the dry powder.

Particle size of dry powders for inhalation affects location of delivery to the respiratory system following inhalation. Suitable particle sizes for pulmonary administration include 0.5 and 10 μm. Microparticles having a diameter of between 0.5 and 10 μm can reach the lungs, successfully passing most of the natural barriers. Particles can also be less than 10 μm in diameter, and less than 5 μm. Exemplary particle sizes that can reach the pulmonary alveoli range from 0.5 μm to 5.8 μm in diameter. Such particles can reach the pulmonary capillaries and can avoid extensive contact with the peripheral tissue in the lung. In one embodiment, dry powder compositions for pulmonary inhalation include microparticles wherein from 35% to 75% of the microparticles have an aerodynamic diameter of between 0.5 and 10 μm or less than 5.8 μm.

In certain embodiments, excipients are selected to reduce or prevent aggregation of particles, and as a result, improve the dispersibility of the powder.

Dry powders for inhalation can include, for example, amino acids (e.g., leucine, isoleucine), mannitol, lactose, and/or FDKP. FDKP is one preferred diketopiperazine for pulmonary applications. FDKP has the following structure:

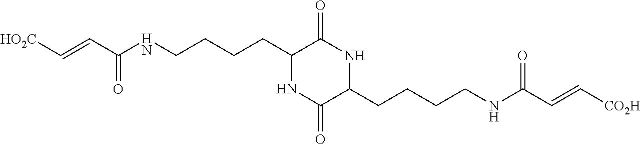 [SMILES: O=C(O)/C=C/C(=O)NCCCCC1NC(=O)C(CCCCNC(=O)/C=C/C(=O)O)NC1=O]

It provides a beneficial microparticle matrix because it has low solubility in acid but is readily soluble at neutral or basic pH. These properties allow FDKP to crystallize and the crystals to self-assemble into formed microparticles under acidic conditions. The particles dissolve readily under physiological conditions where the pH is neutral. Particles with a size range between 0.5 and 10 μm can be readily prepared from FDKP. FDKP microparticles with a specific surface area (SSA) of between 35 and 67 m²/g exhibit characteristics beneficial to delivery of drugs to the lungs such as improved aerodynamic performance and improved drug adsorption. FDKP forms microparticles ideally sized for pulmonary delivery and onto which a cannabinoid (e.g., CBD or THC) can be adsorbed or incorporated. After inhalation, cannabinoid-FDKP inhalation powders dissolve quickly at the surface of the lung, and the cannabinoid is absorbed into the systemic circulation. Cannabinoid-FDKP inhalation powders are expected to have an almost immediate onset of action and improved bioavailability compared to traditional oral products due to avoiding first pass metabolism by the liver.

FDKP is a chiral molecule having trans and cis isomers with respect to the arrangement of the substituents on the substituted carbons on the DKP ring. More robust aerodynamic performance and consistency of particle morphology can be obtained by confining the isomer content to 45-65% trans. Isomer ratio can be controlled in the synthesis and recrystallization of the molecule. Exposure to base promotes ring epimerization leading to racemization, for example during the removal of protecting groups from the terminal carboxylate groups. However, increasing methanol content of the solvent in this step leads to increased trans isomer content. The trans isomer is less soluble than the cis isomers and control of temperature and solvent composition during recrystallization can be used to promote or reduce enrichment for the trans isomer in this step.

Other diketopiperazine compounds and analogs are known to one of skill in the art and may be used in compositions of the present disclosure. For example, diketopiperazine compounds are described in WO2006023943, WO2009146320, WO2010078373, WO2010144785, WO2010144789, WO2012109256, WO2012174472, WO2012174556, and WO2014144895.

In particular embodiments, the dry powders for inhalation include cannabinoids at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders for inhalation include THC at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders for inhalation include THC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders for inhalation include CBD at 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders for inhalation include CBD at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the dry powders for inhalation include PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 0.5-5% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 5-10% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 10-20% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 20-30% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 30-40% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 40-50% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the dry powders for inhalation include cannabinoids at 25% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

The remaining percentage of the dry powders can include excipients. In some examples, the excipient includes amino acids (e.g., leucine, isoleucine), mannitol, lactose, and/or FDKP.

The dry powder composition may be delivered using any suitable dry powder inhaler (DPI). Dry powder inhalers are known in the art and particularly suitable inhaler systems are described in U.S. Pat. Nos. 7,305,986 and 7,464,706, both entitled “Unit Dose Capsules and Dry Powder Inhaler”. Other dry powder dispersion devices for pulmonary administration of dry powders include those described, for example, in U.S. Pat. Nos. 5,522,385 and 5,388,572 and EP Pat. Nos. 129985, 472598, and 467172. Also suitable for delivering the dry powders of the disclosure are inhalation devices such as the Astra-Draco “TURBUHALER”. This type of device is described in detail in U.S. Pat. Nos. 4,668,281, 4,667,668 and 4,805,811. Other suitable devices include dry powder inhalers such as the Rotahaler® (Glaxo), Discus® (Glaxo), Spiros™ inhaler (Dura Pharmaceuticals), and the Spinhaler® (Fisons).

The dry powder composition may be incorporated into a unit dosage form within a selected inhalation device. The amount depends on various factors and can be determined according to, for example, the condition to be treated, the target population, and the inhalation device. According to some embodiments, the unit dosage weight is between 0.2-40 mg, 0.2-20 mg, 10-40 mg, or 20-40 mg. Convenient methods for unit dose packages with metered doses of dry powder medicament are described, e.g., in WO 97/41031.

For additional information regarding dry powder delivery through inhalation, see Prime et al., Review of Dry Powder Inhalers, 26 Adv. Drug Delivery Rev., pp. 51-58 (1997); and Hickey et al., A new millennium for inhaler technology, 21 Pharm. Tech., n. 6, pp. 116-125 (1997).

(v) Dry Powders for Nasal Administration. Particular embodiments include dry powders prepared as compositions or formulations for nasal administration. Particular embodiments include a dry powder including 5-30% DSPC and/or DPPC and 5-50% cannabinoid or a dry powder including 5-20% DSPC and/or DPPC and 10-30% cannabinoid. Particular embodiments include a dry powder including 5-30% PC and 5-50% THC and/or CBD or a dry powder including 5-20% PC and 10-30% THC and/or CBD. Particular embodiments include a dry powder including 5-30% DSPC and/or DPPC and 5-50% THC and/or CBD or a dry powder including 5-20% DSPC and/or DPPC and 10-30% THC and/or CBD.

In particular embodiments, the dry powder can be combined with a liquid to form a suspension for nasal administration. In particular embodiments, the suspension is an aqueous suspension. In particular embodiments, the dry powder can be formulated as a solid or powder for nasal administration.

In particular embodiments, the nasal formulation may include a preservative, suspending agent, wetting agent, tonicity agent, diluent, or a combination thereof. In particular embodiments, the nasal formulations may include from 0.01% to 90%, from 0.01% to 50%, from 0.01% to 25%, from 0.01% to 10%, or from 0.01% to 5% of a pharmacologically suitable suspending liquid which is physiologically acceptable upon administration intranasally. Pharmacologically suitable liquids include polar solvents, including compounds that contain hydroxyl groups or other polar groups. Polar solvents include: water; aqueous saline solutions with a pharmaceutically acceptable salt(s); alcohols, including ethanol and isopropanol; and glycols including propylene glycol, polyethylene glycol, polypropylene glycol, glycol ether, glycerol, and polyoxyethylene alcohols; or a combination thereof.

The nasal formulations may have a pH of 2.0 to 9.0. In particular embodiments, the nasal formulations may contain a pH buffer. For example, a buffer may include any known pharmacologically suitable buffers which are physiologically acceptable upon administration intranasally. The buffer may be added to maintain the pH of the formulation between 3.0 and 7.0, for example.

Sterility or adequate antimicrobial preservation may be provided as part of the nasal formulations. Processes which may be considered for achieving sterility may include any appropriate sterilization steps known in the art. In particular embodiments, the stabilized cannabinoids are produced under sterile conditions. In particular embodiments, the nasal formulations may be sterile filtered and filled in unit dose vials for use in a nasal spray device. Each unit dose vial may be sterile and is suitably administered without contaminating other vials or the next dose. In particular embodiments, one or more ingredients in the nasal formulation may be sterilized by steam or gamma radiation.

In addition to or in lieu of sterilization, the nasal formulations may contain a pharmaceutically acceptable preservative to minimize the possibility of microbial contamination. Suitable preservatives include those that protect the solution from contamination with pathogenic substances, including phenylethyl alcohol, benzalkonium chloride, benzoic acid, or benzoates such as sodium benzoate. Preserving agents may be present in an amount from 0.01% to 1%, or from 0.002% to 0.02% by total weight or volume of the composition.

The nasal formulations may also include from 0.01% to 90%, from 0.01% to 50%, from 0.01% to 25%, from 0.01% to 10%, or from 0.01% to 1% w/w of an emulsifying agent, a wetting agent, or a suspending agent. Such agents may include: polyoxyethylene sorbitan fatty esters or polysorbates, including polyethylene sorbitan monooleate (polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of poly-condensed fatty acids of castor oil; propane-1,2-diol esters of fatty acids; sodium stearoyl-2-lactylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerized fatty acids of soya bean oil; oxidatively polymerized soya bean oil; and pectin extract.

In particular embodiments, the formulations of the present disclosure may include one or more conventional diluents known in the art. A preferred diluent includes water.

Tonicity agents may include sodium chloride, potassium chloride, zinc chloride, calcium chloride, and a combination thereof. Other osmotic adjusting agents include mannitol, glycerol, dextrose, and a combination thereof.

The nasal formulations may further include other excipients and additives which are pharmacologically suitable. The excipients and additives may include surfactants other than phospholipids used in stabilization of cannabinoids as described herein, moisturizers, stabilizers, complexing agents, humectants, antioxidants, or other additives known in the art. Complexing agents include ethylenediaminetetraacetic acid (EDTA) or a salt thereof, such as the disodium salt, citric acid, nitrilotriacetic acid and the salts thereof; and sodium edetate. Pharmaceutically-acceptable humectants, which may inhibit drying of the mucous membrane and prevent irritation, include sorbitol, propylene glycol, polyethylene glycol, glycerol or a combination thereof.

The present compositions nasal administration may be packaged in any conventional manner, such as a nasal applicator. The nasal application may deliver a fixed or non-fixed dose of the stabilized cannabinoid. Containers for various types of nasal formulations are known to one of skill in the art. The medium containing the stabilized cannabinoid and other appropriate ingredients may be contained in a small bottle or similar container, from which it can be dispersed as a mist to be directed into each nostril. Using ambient air as the propelling agent, one may have the bottle made of a flexible plastic, so that merely squeezing the bottle's sides impels the spray out through the nozzle into the nasal cavity. Air may also be the propelling agent for a pump sprayer, in which the user manipulates a small pump button which pumps air into the container and causes the liquid spray to be emitted on the return stroke. Alternatively, the bottle can be pressurized with a gas which is inert to the user and to the ingredients of the solution. The gas may be dissolved under pressure in the container or may be generated by dissolution or reaction of a solid material which forms the gas as a product of dissolution or as a reaction product. Typical gases which can be used include nitrogen, argon, and carbon dioxide. Also, when the formulation is administered as a spray or aerosol, the formulation may be contained in a pressurized container with a liquid propellant including dichlorodifluoro methane or chlorotrifluoro ethylene, among other propellants.

The nasal formulations may be placed in an appropriate atomizing device, e.g., in a pump-atomizer or the like. The atomizing device may be provided with appropriate means for delivery of aqueous spray to the naris. In particular embodiments, the atomizing device is provided with means ensuring delivery of a substantially fixed volume of composition/actuation (i.e. per spray-unit). In particular embodiments, the device administers a metered dosage.

The nasal formulations may be administered into the nose in the form of drops, or any other method which results in topical application to the nasal mucosa. The form of dosage for intranasal administration may include, solutions, suspensions, or emulsions of the stabilized cannabinoid in a liquid carrier in the form of nose drops. Suitable liquid carriers include water, propylene glycol and other pharmaceutically acceptable alcohols. For administration in drop form formulations may suitably be put in a container provided with a conventional dropper/closure device, e.g., including a pipette or the like, delivering a volume of composition/drop. The dosage forms may be sterilized. The dosage forms may also contain adjuvants such as preservatives, stabilizers, emulsifiers or suspending agents, wetting agents, salts for varying the osmotic pressure or buffers.

The nasal formulations may be administered in the form of a powder. For example, a powder nasal composition can be directly used as a powder for a unit dosage form. If desired, the powder can be filled in capsules such as hard gelatin capsules. The contents of the capsule or single dose device may be administered using e.g., an insufflator.

(vi) Dry Powders for Oral Compositions. Particular embodiments include dry powders prepared as oral compositions. Exemplary oral compositions include: capsules; coated tablets; pellets; pills; rapidly-dissolving tablets; sachets; tablets; compositions that can be delivered in a lingually, sublingually, buccally, or oral mucosal or oral epithelial manner; and other forms of oral compositions formed utilizing a dry powder composition as described herein.

In particular embodiments, oral compositions include carriers such as modified amino acids, a surfactant other than phospholipids used in stabilization of cannabinoids as described herein, a detergent, an azone, a pyrrolidone, a glycol, or a bile salt. An amino acid is any carboxylic acid having at least one free amine group and includes naturally occurring, non-naturally occurring and synthetic amino acids. Poly amino acids are either peptides or two or more amino acids linked by a bond formed by other groups which can be linked, e.g. an ester, anhydride, or an anhydride linkage. Peptides are two or more amino acids joined by a peptide bond. Peptides can vary in length from dipeptides with two amino acids to poly peptides with several hundred amino acids. See Chambers Biological Dictionary, editor Peter M. B. Walker, Cambridge, England: Chambers Cambridge, 1989, page 215. Di-peptides, tri-peptides, tetra-peptides, and penta-peptides can also be used.

Carriers which are modified amino acids include acylated fatty acid amino acids (FA-aa) or a salt thereof, which are typically prepared by modifying the amino acid or an ester thereof by acylation or sulfonation. Acylated fatty acid amino acids include N-acylated FA-aa or an amino acid acylated at its alpha amino group with a fatty acid.

Exemplary N-acylated fatty amino acid salts include SNAC. Other names for SNAC include sodium-N-salicyloyl-8-aminocaprylate, monosodium 8-(N-salicyloylamino) octanoate, N-(salicyloyl)-8-aminooctanoic acid monosodium salt, monosodium N-{8-(2-hydroxybenzoyl)amino}octanoate, sodium 8-[(2-hydroxybenzoyl)amino]octanoate, and salcaprozate sodium. SNAC has the structure:

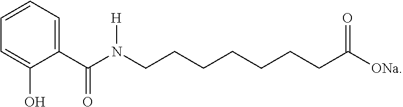

Salts of SNAC may also be used as a carrier.

Other forms of SNAC include:

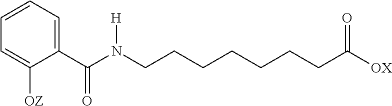

wherein X and Z are independently hydrogen, a monovalent cation, a divalent metal cation, or an organic cation. Examples of monovalent cations include sodium, potassium, and ammonium. Examples of divalent cations include calcium and magnesium. Examples of organic cations include tetramethylammonium.

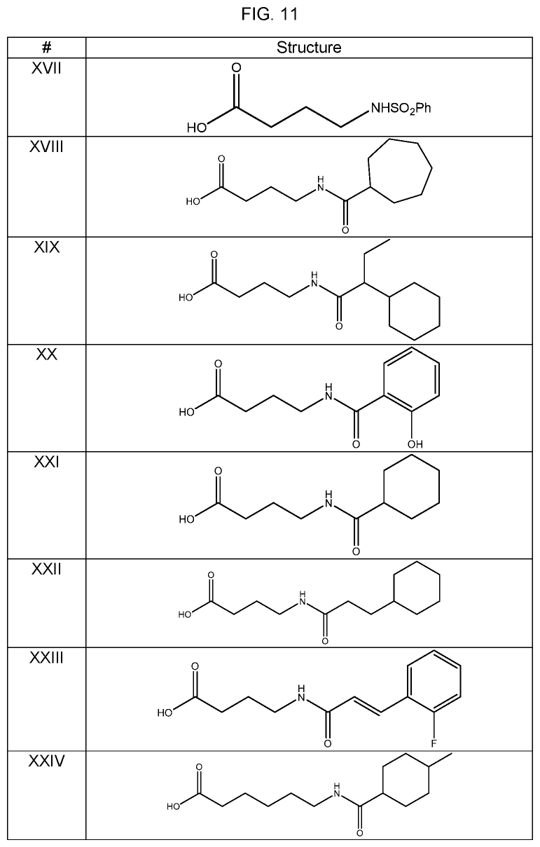

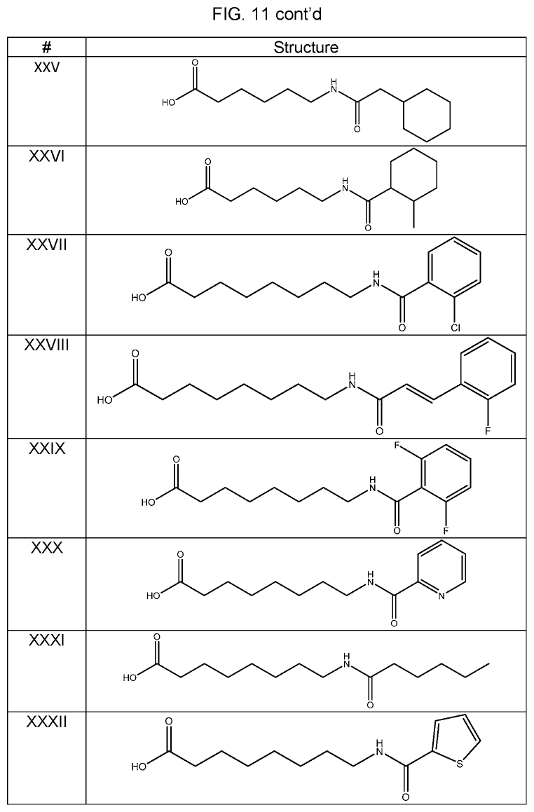

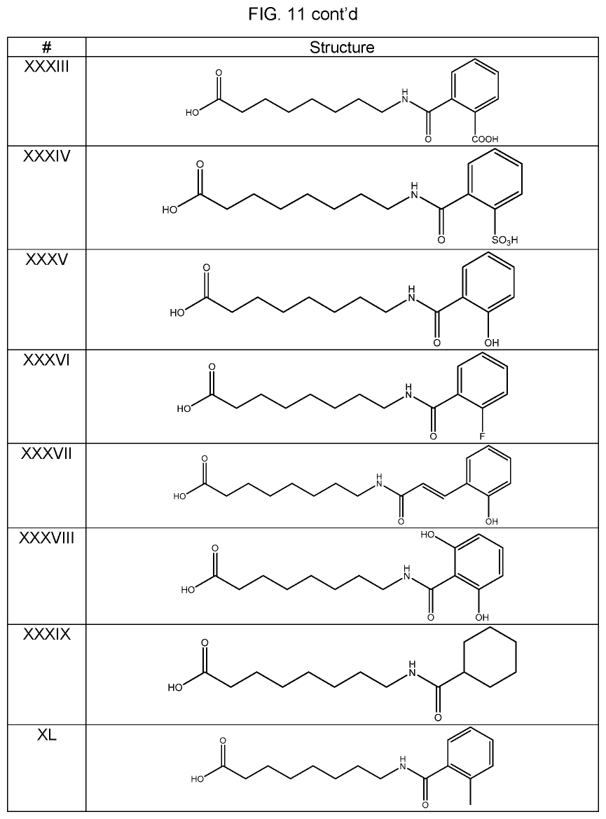

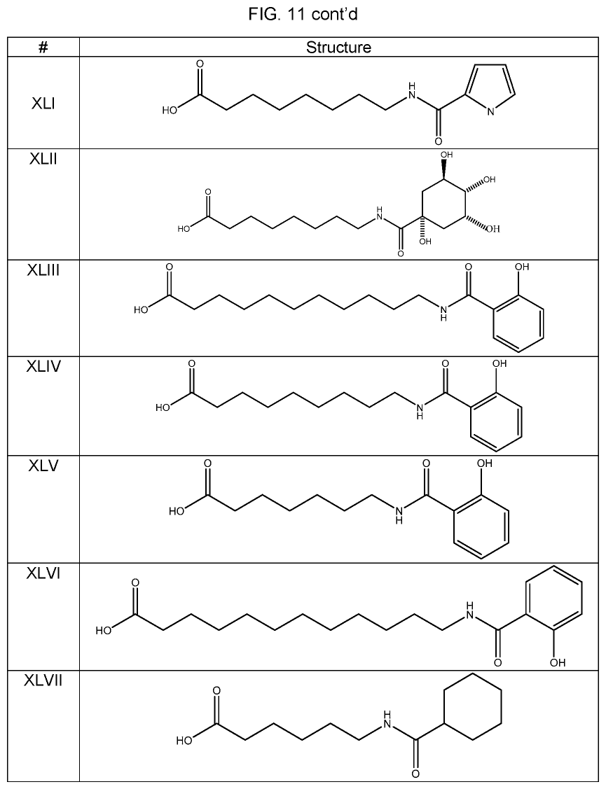

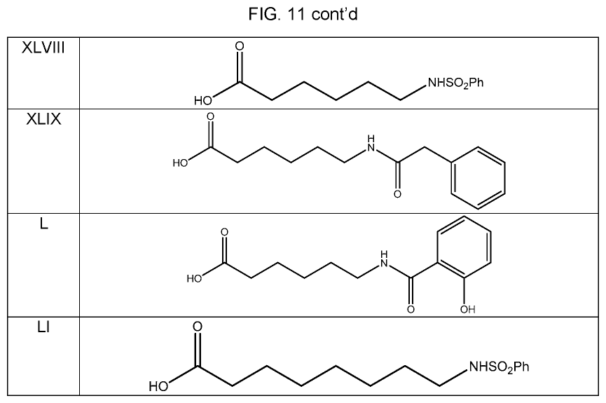

Exemplary modified amino acids, such as N-acylated FA-aas, are provided as compounds XVII-LI (see FIG. 11) and formula LIII. Salts of these compounds and other N-acylated FA-aa can also be used as carriers.

Many of the compounds can be readily prepared from amino acids by methods within the skill of those in the art. For example, compounds XVII-XXIII are derived from aminobutyric acid.

Compounds XXIV-XXVI and XLVII-L are derived from aminocaproic acid. Compounds XXVII-XLII and LI are derived from aminocaprylic acid. For example, the modified amino acid compounds above may be prepared by reacting the single amino acid with the appropriate modifying agent which reacts with free amino moiety present in the amino acids to form amides. Protecting groups may be used to avoid unwanted side reactions as would be known to those skilled in the art.

The amino acid can be dissolved in aqueous alkaline solution of a metal hydroxide, e.g., sodium or potassium hydroxide, and heated at a temperature ranging between 5° C. and 70° C., preferably between 10° C. and 40° C., for a period ranging between 1 hour and 4 hours, preferably 2.5 hours. The amount of alkali employed per equivalent of N H₂ groups in the amino acid generally ranges between 1.25 and 3 mmole, preferably between 1.5 and 2.25 mmole per equivalent of NH₂. The pH of the solution generally ranges between 8 and 13, preferably ranging between 10 and 12.

Thereafter, the appropriate amino acid modifying agent is added to the amino acid solution while stirring. The temperature of the mixture is maintained at a temperature generally ranging between 5° C. and 70° C., preferably between 10° C. and 40° C., for a period ranging between 1 and 4 hours. The amount of amino acid modifying agent employed in relation to the quantity of amino acid is based on the moles of total free NH₂ in the amino acid. In general, the amino acid modifying agent is employed in an amount ranging between 0.5 and 2.5 mole equivalents, preferably between 0.75 and 1.25 mole equivalents, per molar equivalent of total NH₂ group in the amino acid.

The reaction is quenched by adjusting the pH of the mixture with a suitable acid, e.g., concentrated hydrochloric acid, until the pH reaches between 2 and 3. The mixture separates on standing at room temperature to form a transparent upper layer and a white or off-white precipitate. The upper layer is discarded, and the modified amino acid is collected from the lower layer by filtration or decantation. The crude modified amino acid is then dissolved in water at a pH ranging between 9 and 13, preferably between 11 and 13. Insoluble materials are removed by filtration and the filtrate is dried in vacuo. The yield of modified amino acid generally ranges between 30 and 60%, and usually can be 45%.

If desired, amino acid esters, such as, for example benzyl, methyl, or ethyl esters of amino acid compounds, may be used to prepare the modified amino acids. The amino acid ester, dissolved in a suitable organic solvent such as dimethylformamide, pyridine, or tetrahydrofuran can be reacted with the appropriate amino acid modifying agent at a temperature ranging between 5° C. and 70° C., preferably at 25° C., for a period ranging between 7 and 24 hours. The amount of amino acid modifying agent used relative to the amino acid ester is the same as described above for amino acids. This reaction may be carried out with or without a base such as, for example, triethylamine or diisopropylethylamine.

Thereafter, the reaction solvent is removed under negative pressure and the ester functionality is removed by hydrolyzing the modified amino acid ester with a suitable alkaline solution, e.g. 1 N sodium hydroxide, at a temperature ranging between 50° C. and 80° C., preferably at 70° C., for a period of time sufficient to hydrolyze off the ester group and form the modified amino acid having a free carboxyl group. The hydrolysis mixture is then cooled to room temperature and acidified, e.g. aqueous 25% hydrochloric acid solution, to a pH ranging between 2 and 2.5. The modified amino acid precipitates out of solution and is recovered by conventional means such as filtration or decantation. Benzyl esters may be removed by hydrogenation in an organic solvent using a transition metal catalyst.

The modified amino acid may be purified by recrystallization or by fractionation on solid column supports. Suitable recrystallization solvent systems include acetonitrile, methanol and tetrahydrofuran. Fractionation may be performed on a suitable solid column supports such as alumina, using methanol/n-propanol mixtures as the mobile phase; reverse phase column supports using trifluoroacetic acid/acetonitrile mixtures as the mobile phase; and ion exchange chromatography using water as the mobile phase. When anion exchange chromatography is performed, preferably a subsequent 0-500 mM sodium chloride gradient is employed.

In particular embodiments, modified amino acids having the formula

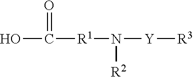

wherein Y is

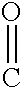

### or SO₂;

R¹ is C₃-C₂₄ alkylene, C₂-C₂₀ alkenylene, C₂-C₂₀ alkynylene, cycloalkylene, or an aromatic, such as arylene;
R² is hydrogen, C₁-C₄ alkyl, or C₂-C₄ alkenyl; and
R³ is C₁-C₇ alkyl, C₃-C₁₀ cycloalkyl, aryl, thienyl, pyrrolo, or pyridyl, and
R³ is optionally substituted by one or more C₁-C₅ alkyl group, C₂-C₄ alkenyl group, F, C, OH, OR¹, SO₂, COOH, COOR¹ or, SO₃H;
may be prepared by reacting in water and the presence of a base, a lactam having the formula

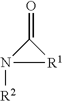

with a compound having the formula R³—Y—X, wherein Y, R¹, R², and R³ are as above and X is a leaving group. A lactam as shown in the above formula can be prepared, for example by the method described in Olah et al., Synthesis, 537-538 (1979).

In particular embodiments, modified amino acids also include an amino acid acylated at its alpha amino group with a fatty acid, which can be represented by the general formula A-X, wherein A is the alpha-amino acid residue and X is a fatty acid attached by acylation to A's alpha-amino group. The amino acids include cationic and non-cationic amino acids. In particular embodiments the term “non-cationic amino acid” refers to an amino acid selected from the group consisting of non-polar hydrophobic amino acids, polar non-charged amino acids, and polar acidic amino acids. In particular embodiments the term “non-cationic amino acid” as used herein refers to amino acids selected from the group consisting of Alanine (Ala), Valine (Val), Leucine (Leu), Isoleucine (lie), Phenylalanine (Phe), Tryptophane (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp), and Glutamic acid.

In particular embodiments, the acylated FA-aa includes an alpha amino acid residue of a non-polar hydrophobic amino acid. In particular embodiments, the acylated FA-aa may be represented by the general formula A-X, wherein A is the amino acid residue of a non-polar hydrophobic amino acid and X is a fatty acid attached by acylation to A's alpha-amino group. In particular embodiments the term “non-polar hydrophobic amino acid” as used herein refers to categorization of amino acids used by the person skilled in the art. In particular embodiments the term “non-polar hydrophobic amino acid” refers to an amino acid selected from the group consisting of Alanine (Ala), Valine (Val), Leucine (Leu), Isoleucine (lie), Phenylalanine (Phe), Tryptophane (Trp), Methionine (Met), Proline (Pro) and Sarcosine.

In particular embodiments, the acylated FA-aa includes the amino acid residue of a polar non-charged amino acid. In particular embodiments the acylated FA-aa may be represented by the general formula A-X, wherein A is the amino acid residue of a polar non-charged amino acid and X is a fatty acid attached by acylation to A's alpha-amino group. In particular embodiments the term “polar non-charged amino acid” as used herein refers to categorization of amino acids used by the person skilled in the art. In particular embodiments the term “polar non-charged amino acid” refers to an amino acid selected from the group consisting of Glycine (Gly), Serine (Ser), Threonine (Thr), Cysteine (Cys), Tyrosine (Tyr), Asparagine (Asn), and Glutamine (Gln).

In particular embodiments, the acylated FA-aa includes the amino acid residue of a polar acidic amino acid. In particular embodiments, the acylated FA-aa may be represented by the general formula A-X, wherein A is the amino acid residue of a polar acidic amino acid and X is a fatty acid attached by acylation to A's alpha-amino group. In particular embodiments, the term “polar acidic amino acid” as used herein refers to categorization of amino acids used by the person skilled in the art. In particular embodiments, the term “polar acidic amino acid” refers to an amino acid selected from the group consisting of Aspartic acid (Asp) and Glutamic acid (Glu).

In particular embodiments, the amino acid residue of the acylated FA-aa includes the amino acid residue of an amino acid that is not encoded by the genetic code. Modifications of amino acids by acylation may be readily performed using acylation agents known in the art that react with the free alpha-amino group of the amino acid.

In particular embodiments, the alpha-amino acids or the alpha-amino acid residues herein are in the L-form unless otherwise stated.

In particular embodiments, the amino acid residue is in the free acid form and/or a salt thereof, such as a sodium (Na+) salt thereof.

Exemplary embodiments of acylated FA-aas may be represented by the general Fa-aa formula LIII:

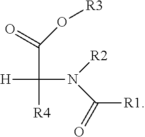

wherein R1 is an alkyl or aryl group including 5 to 19 carbon atoms; R2 is H (i.e. hydrogen), CH₃ (i.e. methyl group), or covalently attached to R4 via a (CH₂)₃ group; R3 is H or absent; and R4 is an amino acid side chain or covalently attached to R2 via a (CH₂)₃ group; or a salt thereof.

The FA-aa can be acylated with a fatty acid including a substituted or unsubstituted alkyl group consisting of 5 to 19 carbon atoms. In particular embodiments, the alkyl group consists of 5 to 17 carbon atoms. In particular embodiments, the alkyl group consists of 5-15 carbon atoms. In particular embodiments the alkyl group consists of 5-13 carbon atoms. In particular embodiments the alkyl group consists of 6 carbon atoms.

In particular embodiments, the acylated FA-aa is soluble at intestinal pH values, particularly in the range pH 5.5 to 8.0, such as in the range pH 6.5 to 7.0. In particular embodiments, the acylated FA-aa is soluble below pH 9.0.

In particular embodiments, the acylated FA-aa has a solubility of at least 5 mg/mL. In particular embodiments, the acylated FA-aa has a solubility of at least 10 mg/mL. In particular embodiments, the acylated FA-aa has a solubility of at least 20 mg/mL. In particular embodiments, the acylated FA-aa has a solubility of at least 30 mg/mL. In particular embodiments, the acylated FA-aa has a solubility of at least 40 mg/mL. In particular embodiments, the acylated FA-aa has a solubility of at least 50 mg/mL. In particular embodiments, the acylated FA-aa has a solubility of at least 60 mg/mL. In particular embodiments, the acylated FA-aa has a solubility of at least 70 mg/mL. In particular embodiments, the acylated FA-aa has a solubility of at least 80 mg/mL. In particular embodiments, the acylated FA-aa has a solubility of at least 90 mg/mL. In particular embodiments, the acylated FA-aa has a solubility of at least 100 mg/mL. In particular embodiments, solubility of the acylated FA-aa is determined in an aqueous solution at a pH value 1 unit above or below pKa of the FA-aa at 37° C. In particular embodiments, solubility of the acylated FA-aa is determined in an aqueous solution at pH 8 at 37° C. In particular embodiments, solubility of the acylated FA-aa is determined in an aqueous solution at a pH value 1 unit above or below pI (isoelectric point) of the FA-aa at 37° C. In particular embodiments, solubility of the acylated FA-aa is determined in an aqueous solution at a pH value 1 unit above or below pI of the FA-aa at 37° C., wherein said FA-aa has two or more ionizable groups with opposite charges. In particular embodiments, solubility of the FA-aa is determined in an aqueous 50 mM sodium phosphate buffer, pH 8.0 at 37° C.

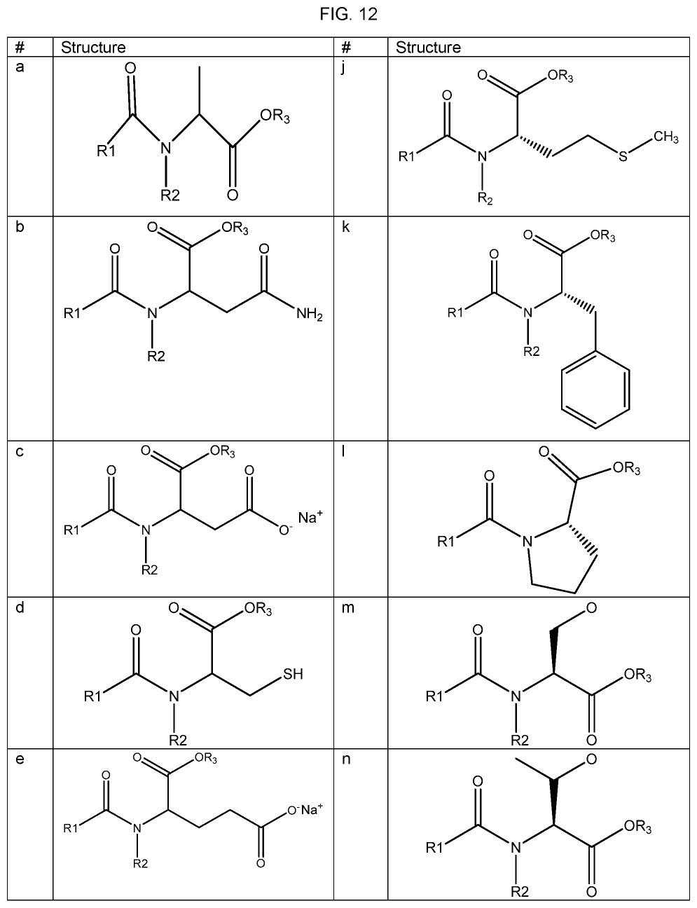

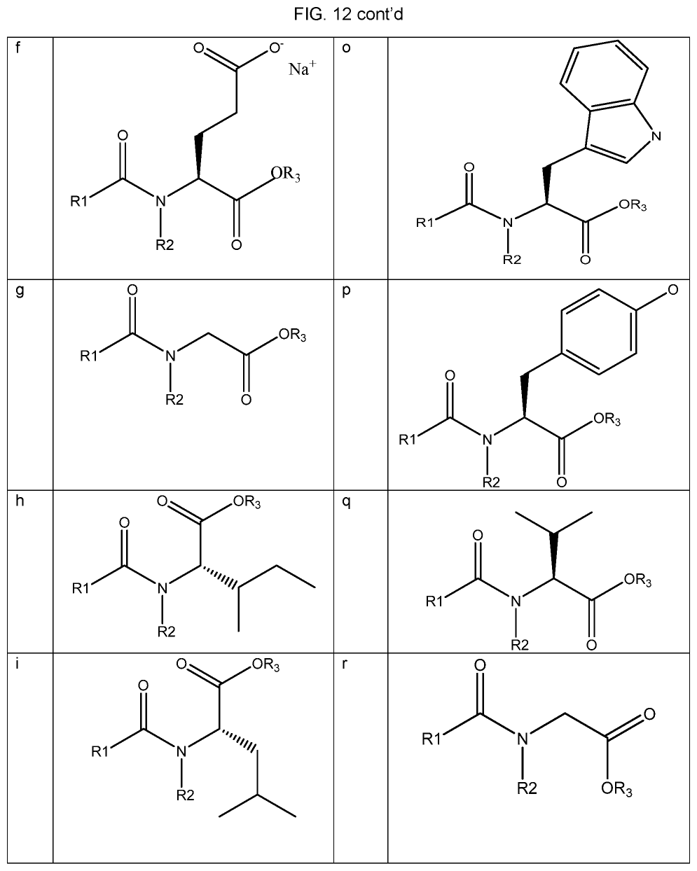

In particular embodiments the acylated FA-aa is selected from the group consisting of formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH₃ (i.e. methyl group), and R3 is H; or a salt or the free acid form thereof. Formulas (a), (b), (c), (d), (e), (f), (g), (h), (i), (j), (k), (l), (m), (n), (o), (p), (q), and (r) are provided in FIG. 12.

In particular embodiments, the acylated FA-aa can be selected from one or more of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophane, sodium N-dodecanoyl valinate, N-dodecanoyl-L-valine, sodium N-dodecanoyl sarcosinate, N-dodecanoyl-L-sarcosine, sodium N-oleoyl sarcosinate, sodium N-decyl leucine, sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decanoyl isoleucinate, N-decanoyl-L-isoleucine, sodium N-decanoyl methioninate, N-decanoyl-L-methionine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophane, sodium N-decanoyl sarcosinate, N-decanoyl-L-Sarcosine, N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl tyrosinate, N-decanoyl-L-tyrosine, sodium N-dodecanoyl asparaginate, sodium N-dodecanoyl glutamic acid, N-dodecanoyl-L-glutamic acid, sodium N-decanoyl glutamic acid, N-decanoyl-L-glutamic acid, Amisoft HS-11 P (sodium Stearoyl Glutamate, Amisoft MS-11 (sodium Myristoyl Glutamate), Amisoft LS-11 (sodium Dodecanoyl Glutamate), Amisoft CS-11 (sodium Cocoyl Glutamate), sodium N-cocoyl glutamate, Amisoft HS-11 P (sodium N-stearoyl glutamate), Amisoft MS-11 (sodium N-myristoyl glutamate), and Amisoft LS-11 (sodium N-dodecanoyl glutamate).

The following acylated FA-aas are commercially available:

| | | Provider (per |
|---|---|---|
| Brand Name | Chemical Name | 14 APR. 2011) |
| Hamposyl L-95 | sodium N-dodecanoyl sarcosinate | Chattem |
| | | Chemicals |
| Hamposyl O | sodium N-oleoyl sarcosinate | Chattem |
| | | Chemicals |
| Hamposyl C | sodium N-cocoyl sarcosinate | Chattem |
| | | Chemicals |
| Hamposyl L-30 | sodium N-dodecanoyl sarcosinate | Chattem |
| | | Chemicals |
| Amisoft HS-11 P | sodium N-stearoyl glutamate | Ajinomoto |
| Amisoft LS-11 | sodium N-dodecanoyl glutamate | Ajinomoto |
| Amisoft CS-11 | sodium N-cocoyl glutamate | Ajinomoto |
| Amisoft MS-11 | sodium N-myristoyl glutamate | Ajinomoto |
| Amilite GCS-11 | sodium N-cocoyl glycinate | Ajinomoto |

In particular embodiments the terms “fatty acid N-acylated amino acid”, “fatty acid acylated amino acid”, “N-acylated fatty amino acid”, or “acylated amino acid” are used interchangeably herein and refer to an amino acid that is acylated with a fatty acid at its alpha-amino group.

Particular embodiments utilize cannabinoids with low solubility, or very low solubility. Particular embodiments utilize cannabinoids that are essentially water insoluble. In particular embodiments, solubility in water is defined as low to zero by the United States pharmacopeia (USP 32-NF 27, 2008) according to the amount of water necessary for the dissolution of one part of solute: Low solubility: 100 to 1000 parts of water necessary for dissolution of one part of solute; very low solubility: 1000 to 10 000 parts of water necessary; essentially water insoluble more than 10 000 parts of water necessary. In particular embodiments, solubility in water or aqueous solubility of a compound refers to the maximum amount of a compound (i.e., the solute) that can be dissolved in a given volume of water at neutral pH and at 25° C.-30° C. Neutral pH can include a pH of 6 to a pH of 8, or a pH of 6.5 to 7.8, or a pH of 6.8 to 7.5, or a pH of 6, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In particular embodiments, solubility in water of a compound at neutral pH can be found in a reference known to one of skill in the art, such as the Merck Index. In particular embodiments, very low solubility can refer to a solubility in water or in an aqueous solution of less than 1 mg/ml, less than 0.1 mg/ml, or less than 0.01 mg/ml at neutral pH and at 25° C.-30° C.

In particular embodiments, N-acylated fatty amino acids act as absorption enhancing agents, thereby creating an administration benefit. Absorption enhancing agents refer to compounds that promote gastrointestinal absorption. Absorption enhancing agents can improve drug absorption by improving the solubility of the drug in the gastrointestinal tract or by enhancing membrane penetration, as compared to a formulation that does not include the absorption enhancing agents. Additional examples of absorption enhancing agents include surfactants other than phospholipids used in stabilization of cannabinoids as described herein, detergents, azones, pyrrolidones, glycols or bile salts.

In particular embodiments, N-acylated fatty amino acids act as bioavailability enhancing agents. Bioavailability refers to the fraction of active ingredient that is actually absorbed by a subject and reaches the bloodstream. In particular embodiments, bioavailability enhancing agents increase the fraction of active ingredient in the bloodstream or result in detection of active ingredient in the bloodstream earlier in time, as compared to a formulation that does not include the bioavailability enhancing agent.

Embodiments utilizing absorption enhancing agents and/or bioavailability enhancing agents (e.g., and in particular embodiments, N-acylated fatty amino acids) can be beneficial because many oral cannabinoid-based compositions designed to address various physiological conditions are inadequate because they are characterized by a delayed onset of action, and low bioavailability. Delayed onset of action presents challenges in clinical indications that require rapid therapeutic effect (e.g. pain and migraine); and low bioavailability requires patients to ingest significantly higher doses than would be required by alternative dosing forms (e.g. smoking, vaping). Particular embodiments disclosed herein provide cannabinoid-based oral compositions with improved stability and bioavailability.

Exemplary excipient classes for oral compositions include binders, buffers, chelators, coating agents, colorants, complexation agents, diluents (i.e., fillers), disintegrants, emulsifiers, flavoring agents, glidants, lubricants, preservatives, releasing agents, surfactants other than phospholipids used in stabilization of cannabinoids as described herein, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the oral compositions to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of oral compositions. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin.

Disintegrants also may be included in the oral compositions in order to facilitate dissolution. Disintegrants, including permeabilizing and wicking agents, are capable of drawing water or saliva up into the oral compositions which promotes dissolution from the inside as well as the outside of the oral compositions. Such disintegrants, permeabilizing and/or wicking agents that may be used include: starches, such as corn starch, potato starch, pre-gelatinized and modified starches thereof; cellulosic agents, such as Ac-di-sol, microcrystalline cellulose, croscarmellose sodium, hydroxymethylcellulose, hydroxypropylcellulose, and hydroxyopropylmethylcellulose; montmorrilonite clays; cross-linked PVP; sweeteners; bentonite; alginates; sodium starch glycolate; gums, such as agar, guar, locust bean, karaya, pectin, Arabic, xanthan and tragacanth; silica with a high affinity for aqueous solvents, such as colloidal silica and precipitated silica; polysaccharides such as maltodextrins and beta-cyclodextrins; and polymers, such as carbopol. Dissolution of the oral compositions may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Emulsifiers are surface active substances which promote the suspension of one liquid in another and promote the formation of a stable mixture, or emulsion, of oil and water. Exemplary emulsifiers include acacia, anionic emulsifying wax, calcium stearate, carbomers, ceto stearyl alcohol, cetyl alcohol, cholesterol, diethanolamine, ethylene glycol palmitostearate, glycerin monostearate, glyceryl monooleate, hydroxypropyl cellulose, hypromellose, lanolin hydrous, lanolin alcohols, lecithin, medium-chain triglycerides, methylcellulose, mineral oil, monobasic sodium phosphate, monoethanolamine, nonionic emulsifying wax, oleic acid, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum, and combinations thereof.

Flavorants are natural or artificial compounds used to impart a pleasant flavor and often odor to oral compositions. Exemplary flavorants include natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize oral composition weight variation. Exemplary glidants include silicon dioxide, colloidal or fumed silica, magnesium stearate, calcium stearate, stearic acid, cornstarch, and talc.

Lubricants are substances used in oral compositions that reduce friction during composition compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Preservatives are substances used in compositions to prevent the growth of microorganisms and/or to prevent degradation of the active ingredient. Exemplary preservatives include parabens, chlorobutanol, phenol, thimerosal, methyl p-hydroxybenzoates, propyl p-hydroxybenzoates, and sorbic acid.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

Particular embodiments include swallowable compositions. Swallowable compositions are those that do not readily dissolve when placed in the mouth and may be swallowed whole without chewing or discomfort. U.S. Pat. Nos. 5,215,754 and 4,374,082 describe methods for preparing swallowable compositions. In particular embodiments, swallowable compositions may have a shape containing no sharp edges and a smooth, uniform and substantially bubble free outer coating.

To prepare swallowable compositions, each of the ingredients may be combined in an intimate admixture with a suitable carrier according to conventional compounding techniques. In particular embodiments of the swallowable compositions, the surface of the compositions may be coated with a polymeric film. Such a film coating has several beneficial effects. First, it reduces the adhesion of the compositions to the inner surface of the mouth, thereby increasing the subject's ability to swallow the compositions. Second, the film may aid in masking the unpleasant taste of certain ingredients. Third, the film coating may protect the compositions from atmospheric degradation. Polymeric films that may be used in preparing the swallowable compositions include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In particular embodiments, the oral compositions may include chewable compositions. Chewable compositions are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable compositions with improved mouthfeel. U.S. Pat. No. 5,965,162, describes kits and methods for preparing comestible units which disintegrate quickly in the mouth, especially when chewed.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

Sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Fondant or gums such as gelatin, agar, arabic gum, guar gum, and carrageenan may be added to improve the chewiness of the compositions. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable compositions. By “enhance the texture” it is meant that the alkyl polysiloxane improves one or more of the stiffness, the brittleness, and the chewiness of the chewable composition, relative to the same preparation lacking the alkyl polysiloxane. By “enhance the mouthfeel” it is meant that the alkyl polysiloxane reduces the gritty texture of the chewable composition once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the backbone. Depending upon their grade, they can further include silica gel. Alkyl polysiloxanes are generally viscous oils. Exemplary alkyl polysiloxanes that can be used in swallowable, chewable or dissolvable compositions include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than 90.5% by weight (CH₃)₃—Si{OSi(CH₃)₂}CH₃ in admixture with 4.0% to 7.0% by weight SiO₂.

To prevent the stickiness that can appear in some chewable compositions and to facilitate conversion of the active ingredients to emulsion or suspension upon taking, the compositions may further include emulsifiers such as glycerin fatty acid ester, sorbitan monostearate, sucrose fatty acid ester, and mixtures thereof. In particular embodiments, one or more of such emulsifiers may be present in an amount of 0.01% to 5.0%, by weight of the administered formulations. If the level of emulsifier is lower or higher, in particular embodiments, an emulsification cannot be realized, or wax value will rise.

In addition to those described above, any appropriate fillers and excipients may be utilized in preparing the swallowable, chewable and/or dissolvable compositions or any other oral composition described herein so long as they are consistent with the described objectives. Excipients are commercially available from companies such as Aldrich Chemical Co., FMC Corp, Bayer, BASF, Alexi Fres, Witco, Mallinckrodt, Rhodia, ISP, and others.

Particular embodiments include oral compositions that are absorbed through the mucosal membranes of the oral cavity (i.e. oral transmucosal delivery). A mucosal membrane includes a mucus-coated biological membrane in a body. In particular embodiments, absorption through the mucosal membranes of the oral cavity include lingual (tongue), sublingual (under the tongue), buccal (cheek), gingival (gum), and palatal (relating to the palate) absorption. In particular embodiments, substances absorbed by mucosal membranes of the oral cavity are rapidly absorbed into the systemic circulation and have quicker onset of action as compared to absorption through the digestive tract. An oral composition for oral transmucosal delivery can be in a form of a lozenge, a pill, a tablet, a capsule, a membrane, a strip, a liquid, a patch, a film, a gel, a spray, a gum, or other form.

A composition for oral transmucosal delivery may include a stabilized cannabinoid and may include: a mucoadhesive that provides for adherence to the mucosa of the mouth of a subject; a binder as described herein; a hydrogel forming excipient; a bulking agent as described herein; a lubricant as described herein; an absorption enhancer as described herein; a buffering excipient as described herein; and coatings and other excipients and factors that modify and control the dissolution time and kinetics of a stabilized cannabinoid.

Exemplary mucoadhesive materials can include: synthetic or biological polymers; and lipids. Examples of natural and/or synthetic polymers include: cellulosic derivatives (such as methylcellulose, carboxymethyl cellulose, hydroxyethyl cellulose, and hydroxyethylmethyl cellulose); natural gums (such as guar gum, xanthan gum, locust bean gum, karaya gum, veegum); polyacrylates (such as carbopol and polycarbophil); alginates, polyoxyethylenes, polyethylene glycols (PEG) of all molecular weights (preferably between 1000 and 40,000 Da, of any chemistry, linear or branched); dextrans of all molecular weights (preferably between 1000 and 40,000 Da of any source); block copolymers, such as those prepared by combinations of lactic & glycolic acid (PLA, PGA, PLGA of various viscosities, molecular weights and lactic-to-glycolic acid ratios); polyethylene glycol-polypropylene glycol block copolymers of any number and combination of repeating units (such as Pluronics, Tektronix or Genapol block copolymers); and a combination of the above copolymers with units either physically or chemically linked (for example PEG-PLA or PEG-PLGA copolymers). A composition for oral transmucosal delivery may contain one or more different mucoadhesives in any combination.

Hydrogel forming excipients that may be present in a composition for oral transmucosal delivery include: polyethylene glycols and other polymers having an ethylene glycol backbone, whether homopolymers or cross linked heteropolymers; block copolymers using ethylene glycol units, such as polyoxyethylene homopolymers (such as POLYOX™ N10/MW=100,000; POLYOX™ N80/MW=200,000; POLYOX™ 1105/MW=900,000; POLYOX™ N-12K/MW=1,000,000; POLYOX™ N-60K/MW=2,000,000; POLYOX™ 301/MW=4,000,000; POLYOX™ 303/MW=7,000,000, DuPont Nutrition Biosciences ApS, Denmark); hydroxypropylmethylcellylose (HPMC) of all molecular weights and grades (such as Metolose® 90SH50000 and Metolose® 90SH30000, Shin-Etsu, Japan), poloxamers (such as Lutrol® F-68, Lutrol® F-127, and Lutrol®F-105, BASF Corporation, Florham Park, NJ), polyethylene glycol monoalkyl ether; polyethylene glycols (PEG, such as PEG-1500, PEG-3500, PEG-4000, PEG-6000, PEG-8000, PEG-12000, and PEG-20,000), natural gums (e.g., xanthan gum, locust bean gum,); cellulose derivatives; polyacrylic acid-based polymers either free or cross-linked, and combinations thereof; and biodegradable polymers (such as polylactic acids, polyglycolic acids and any combination thereof, whether a physical blend or cross-linked). In particular embodiments, the hydrogel components may be cross-linked.

The composition for oral transmucosal drug delivery may also include at least one controlled release modifier which is a substance that upon hydration of the composition will preferentially adhere to the stabilized cannabinoid and thus reduce the rate of the active ingredient diffusion from the oral dosage form. Such excipients may also reduce the rate of water uptake by the composition and thus enable a more prolonged drug dissolution and release from the oral dosage form. In particular embodiments, such controlled release modifiers are capable of binding molecularly to the stabilized cannabinoid via physical (and therefore reversible) interactions, thus increasing the effective molecular weight of the stabilized cannabinoid and thus further modifying its permeation (diffusion) characteristics through the mucosal membranes. In particular embodiments, such controlled release modifiers upon hydration may form concrete structures that may entrap spontaneously the stabilized cannabinoid and thus further prolong its action. Exemplary controlled release modifiers include lipids, sterols, surfactants other than phospholipids used in stabilization of cannabinoids as described herein, polymers, and salts. In general the selected excipient(s) are lipophilic and capable of complexing to the stabilized cannabinoid. The degree of association of the release modifier and the stabilized cannabinoid can be varied by altering the modifier-to-stabilized cannabinoid ratio in the formulation. In addition, such interaction may be appropriately enhanced by the appropriate combination of the release modifier with the stabilized cannabinoid in the manufacturing process. Alternatively, the controlled release modifier may be a charged polymer either synthetic or biopolymer bearing a net charge, either positive or negative, and which is capable of binding to the stabilized cannabinoid via electrostatic interactions thus modifying both its diffusion through the oral dosage form and/or the kinetics of its permeation through the mucosal surface. Such electrostatic interactions may be reversible and do not involve permanent chemical bonds with the stabilized cannabinoid.

The oral transmucosal compositions are typically designed to achieve stabilized cannabinoid dissolution times that may vary from 30 seconds up to 1 minute, 2 minutes, 3 minutes, 4 minutes, 5 minutes, 10 minutes, 15 minutes, 30 minutes, 1 hour, 2 hours, 4 hours, 8 hours or longer, dependent upon the patient and circumstances of active ingredient administration as well as the intrinsic active ingredient pharmacokinetics. It will be understood that the composition of the oral transmucosal formulations may be adjusted to provide for both a range of doses and a range of dissolution times to fit particular clinical situations.

In particular embodiments, the oral compositions include cannabinoids at 0.5%, 0.75%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% %, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the oral compositions include THC at 0.5%, 0.75%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% %, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the oral compositions include THC at 0.5%, 0.75%, 1%, 1.5%, 2%, 2.5%, 3%, 3.5%, 4%, 4.5%, or 5% w/w of the dry powder.

In particular embodiments, the oral compositions include THC at 5%, 6%, 7%, 8%, 9%, 10%, 11%, 12%, 13%, 14%, 15%, 16%, 17%, 18%, 19%, or 20% w/w of the dry powder.

In particular embodiments, the oral compositions include CBD at 0.5%, 0.75%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% %, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w of the dry powder.

In particular embodiments, the oral compositions include CBD at 0.5%, 0.75%, 1%, 1.5%, 2%, 2.5%, 3%, 3.5%, 4%, 4.5%, or 5% w/w of the dry powder.

In particular embodiments, the oral compositions include CBD at 5%, 6%, 7%, 8%, 9%, 10%, 11%, 12%, 13%, 14%, 15%, 16%, 17%, 18%, 19%, or 20% w/w of the dry powder.

In particular embodiments, the oral compositions include PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 0.5-5% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 5-10% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 10-20% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 20-30% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 30-40% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 40-50% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

In particular embodiments, the oral compositions include cannabinoids at 15% w/w of the dry powder and PC at 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w of the dry powder.

The remaining percentage of the oral compositions can include excipients. In some examples, the excipient includes an N-acylated fatty amino acid, such as SNAC. As indicated, numerous additional excipients can also be included.

Oral compositions can be individually wrapped or packaged as multiple units in one or more packages, cans, vials, blister packs, or bottles of any size. Doses are sized to provide therapeutically effective amounts.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990. Moreover, formulations can be prepared to meet sterility, pyrogenicity, general safety, and purity standards as required by U.S. FDA and/or other relevant foreign regulatory agencies.

(vii) Methods of Use. Inhalable and oral compositions disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing therapeutically effective amounts. Therapeutically effective amounts include those that provide effective amounts, prophylactic treatments, and/or therapeutic treatments.

An “effective amount” is the amount of a composition necessary to result in a desired physiological change in a subject. Effective amounts are often administered for research purposes. Representative effective amounts disclosed herein can reduce pain perception in an animal model (neuropathic pain, acute pain, visceral pain), stimulate appetite in an animal model, reduce seizures (e.g., epileptic seizures) in an animal model, reverse bone loss in an animal model, relieve migraine (vasoconstrict cranial blood vessels) in an animal model, treat addiction in an animal model, reduce anxiety in an animal model, and/or reduce symptoms of asthma in an animal model.

A “prophylactic treatment” includes a treatment administered to a subject who does not display signs or symptoms of a disease or nutritional deficiency or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As one example of a prophylactic treatment, a composition disclosed herein can be administered to a subject who is at risk of developing a migraine headache. An effective prophylactic treatment of a migraine headache occurs when the number of migraines per month experienced by a subject is reduced by at least 10% or in particular embodiments, by 25%. Assessments of migraine can include: neuroimaging tests, such as magnetic resonance imaging (MRI) and/or computerized tomography (CT) to rule out intracranial pathology; a migraine disability assessment (MIDAS) questionnaire, which assesses pain frequency, pain intensity, and the impact of migraines on work/school, domestic chores, and leisure activities (Stewart W F. Cephalalgia 1999; 19(2):107-114); a Migraine Interictal Burden Scale (MIBS) questionnaire, which measures interictal (the period between episodes) migraine-related burden in four areas: work/school, family and social life, making plans or commitments, and emotional/affective and cognitive distress (Buse D C. Neurology 2007; 68(suppl 1):A89); and 36- and 12-Item Short-Form Health Surveys (SF-36 and SF-12), which measure Health-Related Quality of Life (HRQoL). For example, subjects with migraines have significantly lower SF-36 scores compared to subjects without migraine (Solomon et al. Headache 1993; 33(7):351-358). In particular embodiments, the impact of migraines on HRQoL can be assessed by a Migraine-Specific Quality of Life Questionnaire (MSQ) version 2.1, which includes 14 items that measure the degree to which migraine affects a subject's daily activities (e.g., work and social activities) and emotions during a 4-week period. Migraines can be classified according to the International Classification of Functioning, Disability, and Health (World Health Organization International Classification of Functioning, Disability and Health. Agenda item 13.9, 54th World Health Assembly, 9th plenary meeting, May 22, 2001, World Wide Web at who.int/classification/icf/site/whares/wha-en.pdf Accessed November 2007).

As another example of a prophylactic treatment, a composition disclosed herein can be administered to a subject who is at risk of developing a headache. An effective prophylactic treatment of headaches occurs when the number, intensity, or duration of headaches is prevented or reduced by at least 5%, by at least 10%, by at least 15%, by at least 20%, by at least 25%, or more. Headaches can be classified as primary (e.g., migraine, tension type, and cluster headache) or secondary, where the headache is a symptom of another disorder, such as meningitis or subarachnoid hemorrhage. Guidelines can be used to rule out secondary causes of headache (Pearls: headache. Dodick D W Semin Neurol. 2010; 30(1):74-81; Perry et al. JAMA. 2013; 310(12):1248-55). Assessment of headache can include: neuroimaging tests to rule out intracranial pathology; and a Headache Impact Test (HIT), which assesses pain severity, fatigue, mood, and impact of headaches on work/school, domestic chores, and leisure activities (Ware J E Med Care 2000; 38(9) (suppl):II73-II82).

As another example of a prophylactic treatment, a composition disclosed herein can be administered to a subject who is at risk of having an epileptic seizure. An effective prophylactic treatment of epileptic seizures occurs when the number of seizures per month is reduced by at least 10% or in particular embodiments, by 25%, as measured by tests, such as ictal scalp electroencephalogram (EEG) or ambulatory EEG monitoring and home video recording. A neurological exam, a blood test, a lumbar puncture, and/or neuroimaging tests (e.g., MRI, CT, positron emission tomography (PET), and/or single-photon emission computerized tomography (SPECT)) can be used to confirm or rule out causes of epileptic seizure such as hemorrhage, infection, tumor and disorders related to cerebrospinal fluid hypertension or hypotension.

As another example of a prophylactic treatment, a composition disclosed herein can be administered to a subject who is at risk of suffering from neuropathic pain. Neuropathic pain refers to pain arising as a direct consequence of a lesion or disease affecting the somatosensory system (Treede et al. J Neurology. 2008; 70(18):1630-1635). Neuropathic pain can be spontaneous (stimulus-independent) or induced by a stimulus (stimulus-dependent or provoked). The stimulus can include a mechanical, a thermal, and/or a chemical stimulus. An effective prophylactic treatment of neuropathic pain occurs when the occurrence of the neuropathic pain is reduced by at least 10%, or in particular embodiments, by 25% as measured by a standard subjective or objective pain assessment. Assessments of neuropathic pain can include: interview questions on a questionnaire including Neuropathic Pain Questionnaire (NPQ) (Krause and Backonja. Clin J Pain. 2003; 19(5):306-314), ID Pain (Portenoy R. Curr Med Res Opin. 2006; 22(8):1555-1565), and PainDETECT (Freynhagen et al. Curr Med Res Opin. 2006; 22(10):1911-1920); and interview questions and physical tests (e.g., pinprick test, tactile test, pain to light touch) included in the Leeds Assessment of Neuropathic Symptoms and Signs (LANSS) scale (Bennett M. Pain 2001; 92(1-2):147-157), Douleur Neuropathique en 4 Questions (DN4) questionnaire (Bouhassira et al. Pain. 2005; 114(1-2):29-36), and the Standardized Evaluation of Pain (StEP) (Scholz et al. PLoS Med. 2009; 6(4):e1000047).

As another example of a prophylactic treatment, a composition disclosed herein can be administered to a subject who is at risk of developing breakthrough pain. Breakthrough pain refers to pain that can spontaneously occur in a chronic condition (e.g, arthritis, back pain, fibromyalgia, multiple sclerosis) and/or pain that breaks through an existing analgesic regimen, commonly experienced in cancer patients and patients with other terminal conditions. Breakthrough pain can include a transitory increase in pain of moderate or severe intensity that occurs on a background of otherwise controlled persistent pain of moderate intensity or less. An effective prophylactic treatment of breakthrough pain occurs when the occurrence of breakthrough pain is reduced by 10%, and in particular embodiments, by 25% by a standard subjective or objective pain assessment. Assessments of breakthrough pain can include: the Breakthrough Pain Questionnaire (BPQ; Portnoy and Hagen. Pain. 1990; 41:273-281); the Alberta Breakthrough Pain Assessment Tool for cancer patients (ABPAT; Hagen et al. J Pain Symptom Manage. 2008; 35(2):136-152); the Breakthrough Pain Assessment Tool (BAT; Webber et al. J Pain Symptom Manage. 2014; 48(4):619-631); the Memorial Pain Assessment Card (MPAC), which measures pain intensity, pain relief, and mood, using visual analog scales and an eight-item verbal rating scale (Fishman et al. Med Clin North Am. 1987:71:271-287); and the Brief Pain Inventory (BPI), which uses a numerical zero to 10 scale (where zero means no pain and 10 means worst pain possible) to measure pain intensity and interference with life activities (Daut et al. Pain. 1983:17:197-210).

As another example of a prophylactic treatment, a composition disclosed herein can be administered to a subject who is at risk of developing chemotherapy induced nausea and vomiting (CINV). An effective prophylactic treatment of CINV occurs when the frequency, severity, and/or duration of CINV is reduced by 10%, and in particular embodiments, by 25% measured by a standard subjective or objective CINV assessment. Assessment of CINV can include: self-report questionnaires including the Morrow Assessment of Nausea and Emesis, which specifically includes questions regarding pretreatment nausea (Morrow G R. Cancer. 1984; 53(10):2267-2278); the MASCC Antiemesis Tool (Multinational Association of Supportive Care in Cancer. A quick guide to the MASCC Antiemesis Tool (MAT). World Wide Web at MASCC.org./mc/page.do?sitePageld=88036); the Index of Nausea, Vomiting, and Retching (INVR; Rhodes and McDaniel. Oncol Nurs Forum. 1999; 26(5):889-894); the Common Toxicity Criteria (Cancer Care Nova Scotia. Guidelines for the management of nausea and vomiting in cancer patients. World Wide Web at cancercare.ns.ca.), which focuses on a subject's ability to maintain oral intake or need for intravenous fluid replacement; the Functional Living Index-Emesis (Decker et al. Support Oncol. 2006; 4(1):35-52); and the Osoba Nausea and Vomiting Module (Martin et al. Cancer. 2003; 9(3):645-655). The Behavioral Observation tool involves nurses assessing a subject's experience and reporting subject behaviors (Chapko et al. J Pain Symptom Manage. 1991; 6(1):15-23). Questionnaires can include questions on duration, frequency, severity, and associated distress of nausea, vomiting, and retching. Some assessment tools use a visual analog scale or numerical rating scale to measure degree of severity of a symptom. Severity and/or distress can be also be assessed using a categorical rating scale, such as none, mild, moderate, or severe.

As an example of a prophylactic treatment of a nutritional deficiency, a composition disclosed herein can be administered to a subject who is at risk of developing rickets from insufficient vitamin C, anemia from insufficient dietary iron, and/or bone loss from insufficient calcium. An effective prophylactic treatment of these conditions occurs when the conditions are avoided or delayed due to nutritional supplementation with a composition disclosed herein.

As another example of a prophylactic treatment, a composition disclosed herein can be administered to a subject who is at risk of developing agitation or delirium. An effective prophylactic treatment of agitation or delirium occurs when delirium incidence is prevented or reduced by at least 5%, by at least 10%, or more as measured by standard subjective or objective agitation or delirium assessments such as the Confusion Assessment Method. In particular embodiments, agitation or delirium includes inattention, cognitive problems, perceptual problems, psychomotor changes, sleep-wake cycle disturbances, emotional disturbances, and/or confusion. In particular embodiments, an effective prophylactic treatment of agitation or delirium occurs when self-destructive or suicidal acts, hallucinations, and/or delusions are minimized as measured by standard subjective or objective agitation or delirium assessments or as measured by laboratory tests in cases where there is an underlying physiological cause such as drug withdrawal, hypoglycemia, stroke, trauma, infection/inflammation, metabolic disorder, or poisoning.

As another example of a prophylactic treatment, a composition disclosed herein can be administered to a subject who is at risk of developing anxiety disorder. An effective prophylactic treatment of anxiety disorder occurs when symptoms such as restlessness; fatigue; difficulty concentrating; irritability or explosive anger; muscle tension; sleep disturbances; and/or personality changes are prevented or reduced in frequency or duration as measured by a standard subjective or objective anxiety disorder assessment. Anxiety disorder can be assessed by a number of tools including: the Subjective Units of Distress Scale (SUDs), a self-assessment tool that measures the intensity of distress or nervousness in people with social anxiety on a scale from 0 to 100 (Benjamin et al. Behav Cogn Psychother. 2010; 38(4):497-504); the Beck Anxiety Inventory (BAI; Beck et al. J Consult Clin Psychol. 1988; 56(6):893-897), focusing on somatic and panic-like symptoms of anxiety; the State-Trait Anxiety Inventory (STAI-T; Julian L J. Arthritis Care Res (Hoboken) 2011; 63(011): 10.1002/acr.20561), focusing on two subscales, current state of anxiety and anxiety proneness; the Hospital Anxiety and Depression Scale-Anxiety (HADS-A; Zigmond and Snaith. Acta Psychiatr Scand. 1983; 67(6):361-370), involving a 4-point Likert scale ranging from 0 to 3 to evaluate common dimensions of anxiety; and the Generalized Anxiety Disorder 7-item scale (GAD-7; Spitzer et al. Arch Intern Med. 2006; 166(10):1092-1097), which includes a short questionnaire that is useful for screening, with a score of 0-4 indicating minimal anxiety, a score of 5-9 indicating mild anxiety, a score of 10-14 indicating moderate anxiety, and a score greater than 15 indicating severe anxiety.

As another example of a prophylactic treatment, a composition disclosed herein can be administered to a subject who is at risk of developing a panic attack or panic disorder. An effective prophylactic treatment of a panic attack or panic disorder occurs when symptoms such as racing heartbeat; sweating; feeling short of breath; and/or feeling fear or dread, or worry about having a panic attack or panic disorder are prevented or reduced in intensity or frequency as measured by a standard subjective or objective panic attack assessment. Tools to assess panic attacks can include: the Patient Health Questionnaire-Panic Disorder (PHQ-PD; Spitzer et al. JAMA. 1999; 282(18):1737-1744), which involve questions that review the history and frequency of panic attacks and relate to somatic symptoms of panic attacks; the Panic Disorder Screener (PADIS; Batterham et al. Psychiatry Research. 2015; 228:72-76), a brief population screening tool, with a cut-off score of 4 or higher indicating criteria for panic disorder; the Panic and Agoraphobia Scale (PAS; Bandelow, B. International Clinical Psychopharmacology 1995; 73-81), measuring severity of panic disorder in subjects with or without agoraphobia, available as clinician-administered or self-rating; and the Panic Disorder Severity Scale (PDSS; Shear et al. American J Psychiatry 1997; 154(11):1571-1575), a brief clinician rating scale that measures the severity of seven dimensions of panic disorders including the frequency of panic attacks, distress during panic attacks, anticipatory anxiety, agoraphobic fear and avoidance, interoceptive fear and avoidance, impairment in work functioning, and impairment in social functioning.

As another example of a prophylactic treatment, a composition disclosed herein can be administered to a subject who is at risk of developing sleep apnea. An effective prophylactic treatment of sleep apnea occurs when symptoms such as snoring; choking, snorting, or gasping during sleep; pauses in breathing during sleep; waking up at night short of breath; and/or daytime sleepiness and fatigue are prevented or reduced in frequency as measured by, for example, a recording of the subject's sleep or by a sleep partner.

As another example of a prophylactic treatment, a composition disclosed herein can be administered to a subject who is at risk of developing alcohol or opioid withdrawal syndrome. In particular embodiments, an effective prophylactic treatment of alcohol withdrawal syndrome occurs when onset and/or duration of symptoms of alcohol withdrawal syndrome such as: anxiety; excess perspiration; tremors, particularly in hands; dehydration; increased heart rate and blood pressure; insomnia; nausea and vomiting; diarrhea; seizures; hallucinations; delirium; extreme fluctuations in body temperature and blood pressure; and/or extreme agitation are prevented or reduced as measured by an assessment tool such as Alcohol Withdrawal Scale (AWS; Nowak H, editor. Nursing education and nursing management of alcohol and other drugs. Sydney: CEIDA; 1989). In particular embodiments, an effective prophylactic treatment of opioid withdrawal syndrome occurs when onset and/or duration of symptoms of opioid withdrawal syndrome such as: nausea and vomiting; anxiety; insomnia; hot and cold flushes; perspiration; muscle cramps; watery discharge from eyes and nose; and/or diarrhea are prevented or reduced as measured by an assessment tool such as Short Opioid Withdrawal Scale (SOWS; Gossop M. The development of a short opiate withdrawal scale. Addictive Behaviors. 1990; 15: 487-490).

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, a composition disclosed herein can be administered to a subject who has a migraine headache. An effective therapeutic treatment of the migraine headache occurs when the severity of the headache is reduced or relieved completely and/or the headache resolves more quickly measured by a standard subjective or objective headache assessment.

As one example of a therapeutic treatment, a composition disclosed herein can be administered to a subject who has a headache. An effective therapeutic treatment of headaches occurs when the number, intensity, or duration of headaches is reduced or relieved completely as measured by a standard subjective or objective headache assessment. Assessments of headaches can include tools described herein.

Another example of a therapeutic treatment includes administration of a composition disclosed herein to a subject experiencing CINV. A therapeutic treatment of CINV occurs when the vomiting is reduced or ceases (or ceases more quickly) and the nausea is relieved measured by a standard subjective or objective CINV assessment. CINV assessments can be conducted as described herein.

Another example of a therapeutic treatment includes administration of a composition disclosed to a subject who has osteoporosis. An effective therapeutic treatment of osteoporosis occurs when bone density has increased by 10% and in particular embodiments, by 25%. Bone density (e.g., of the hip and/or spine) can be determined by dual energy x-ray absorptiometry (DEXA). Bone density results are reported as T-scores. In particular embodiments, according to the World Health Organization, a T-Score of −1.0 or above is normal bone density, a T-score between −1.0 and −2.5 indicates low bone density or osteopenia, and a T-score of −2.5 or below indicates osteoporosis. Screening tools can also be used to evaluate the status of bone density including: simple calculated osteoporosis risk estimation (SCORE; Lydick et al. Am J Manag Care. 1998; 4(1):37-48), involving consideration of age, body weight, race, hormone therapy use, fracture history, and history of rheumatoid arthritis as risk factors; osteoporosis risk assessment instrument (ORAI; Cadarette et al. Canadian Multicentre Osteoporosis Study. JAMA. 2001; 286(1):57-63), involving consideration of age, body weight, and hormone therapy use as risk factors; osteoporosis self-assessment tool (OST; Geusens et al. Mayo Clin Proc. 2002; 77(7):629-637), involving consideration of age and body weight as risk factors; the body weight criterion (BW; Michaëlsson et al. Osteoporos Int. 1996; 6(2):120-126); the osteoporosis index of risk (OSIRIS; Sedrine et al. Gynecol Endocrinol. 2002; 16(3):245-250), involving consideration of age, body weight, hormone therapy, and fracture history as risk factors; and the Age, Body size, No Estrogen tool (ABONE; Weinstein and Ullery. Am J Obstet Gynecol. 2000; 183(3):547-549).

Another example of a therapeutic treatment includes administration of a composition disclosed herein to a subject who has anxiety. An effective therapeutic treatment of anxiety occurs when the severity of the anxiety is reduced or relieved completely and/or more quickly measured by a standard subjective or objective anxiety assessment. Assessments of anxiety can be conducted as described herein.

As one example of a therapeutic treatment, a composition disclosed herein can be administered to a subject who has a panic attack or panic disorder. An effective therapeutic treatment of a panic attack or panic disorder occurs when intensity and/or frequency of the panic attack are reduced or eliminated as measured by a standard subjective or objective panic attack assessment. Assessments of panic attack or panic disorder can be conducted as described herein.

Another example of a therapeutic treatment includes administration of a composition disclosed herein to a subject who has multiple sclerosis (MS). An effective therapeutic treatment of MS occurs when the score in a standard walk test improves by 10% and in particular embodiments, by 25%. Rating scales, performance tests, and patient self-report questionnaires can be used to evaluate walking in MS. Rating scales include: the Kurtzke expanded disability status scale (Kurtzke EDSS; Kurtzke J F. Neurology. 1983; 33(11):1444-1452), which involves a numerical score from 0 to 10, with walking assessed in the middle range of the scale from 4.7 to 7.5, and consideration of maximum distance walked and use of an assistive device; the Hauser Ambulation Index (Hauser et al. N Engl J Med. 1983; 308(4):173-180), which involves a scale from 0 to 9, with scoring dependent on the need for an assistive mobility device and on the ability and time required to walk 25 feet; the Dynamic Gait Index (DGI; Shumway-Cook and Wollacott. Motor Control: Theory and Practical Applications. Baltimore, MD: Williams and Wilkins; 1995) incorporates walking, stair climbing, and balance; and the Rivermead Visual Gait Assessment (RVGA; Lord et al. Clin Rehabil. 1998; 12(2):107-119), a qualitative gait analysis tool that considers deviations during the stance and swing phases of gait, including a total score that can range from 0 to 59, with higher scores indicating more severe deviations from normal. Timed walking tests are objective assessments of MS and can include: a timed 25-foot walk (or a timed walk of another specified distance; Fischer et al. Mult Scler. 1999; 5(4):244-250); the 6-minute walk test, which records the maximum distance walked in 6 minutes (Goldman et al. Mult Scler. 2008; 14(3):383-390); the timed Up and Go test (TUG; Podsiadlo and Richardson. J Am Geriatr Soc. 1991; 39(2):142-148) considers the time required for a person to rise from a chair, walk 3 m, turn around, walk back to the chair, and sit down; and the Six Spot Step Test (SSST; Nieuwenhuis et al. Mult Scler. 2006; 12(4):495-500), which considers lower extremity function.

As one example of a therapeutic treatment of a nutritional deficiency, a composition disclosed herein can be administered to a subject who has rickets from insufficient vitamin C, anemia from insufficient dietary iron, and/or bone loss from insufficient calcium. An effective therapeutic treatment of these conditions occurs when the conditions are reduced or resolved due to nutritional supplementation with a composition disclosed herein.

As one example of a therapeutic treatment, a composition disclosed herein can be administered to a subject who has autism spectrum disorder (ASD). An effective therapeutic treatment of ASD occurs when social communication and interaction are improved and/or repetitive patterns of behavior are reduced or eliminated. As examples, symptoms such as: limited or no verbal or facial communication; no interest in others; repetitive behaviors; echolalia; and extreme sensitivity to noise are improved as measured by a standard subjective or objective ASD assessment. Assessments of ASD include: ages and stages questionnaire that parents can complete, which include questions on gross motor, fine motor, problem-solving, and personal adaptive skills and provides a pass/fail score; Communication and Symbolic Behavior Scales (CSBS), which assesses children up to the 24-month level; the Parents' Evaluation of Developmental Status (PEDS), which is a parent interview form that can be used as a screening and surveillance tool; the Modified Checklist for Autism in Toddlers (MCHAT), a parent-completed questionnaire that identifies children at risk for autism; and the Screening Tool for Autism in Toddlers and Young Children (STAT), an interactive tool including 12 activities that assess play, communication, and imitation skills.

As one example of a therapeutic treatment, a composition disclosed herein can be administered to a subject who has agitation or delirium. An effective therapeutic treatment of the agitation or delirium occurs when inattention, cognitive problems, perceptual problems, psychomotor changes, sleep-wake cycle disturbances, emotional disturbances, and/or confusion is reduced or eliminated as measured by standard subjective or objective agitation or delirium assessments such as the Confusion Assessment Method or as measured by laboratory tests in cases where there is an underlying physiological cause such as drug withdrawal, hypoglycemia, stroke, trauma, infection/inflammation, metabolic disorder, or poisoning.

As one example of a therapeutic treatment, a composition disclosed herein can be administered to a subject who has Dravet Syndrome. An effective therapeutic treatment of Dravet Syndrome occurs when the severity, frequency, and/or duration of convulsive seizures is reduced or eliminated as measured by a standard subjective or objective seizure assessment. Assessments of seizure can include long term video-EEG monitoring; a clinical risk score (Hattori et al. Epilepsia 2008; 49:626-633) that considers the age at seizure onset, total number of seizures before one year of age, total number of prolonged seizures (longer than 10 min), and the seizure type and trigger; and evaluation of symptoms prior to (preictal), during (ictal), and after (postictal) a seizure. Ictal symptoms include aura (subjective sensations), behavior (mood or behavioral changes), vocal (e.g., cry or gasp, slurring of words, garbled speech), motor (head or eye turning, eye deviation, posturing, rhythmic jerking, stiffening, purposeless repetitive movements, lip smacking), respiration (change in breathing pattern, cessation of breathing, cyanosis), autonomic (pupillary dilatation, drooling, change in respiratory or heart rate, incontinence, pallor, vomiting), and/or loss of consciousness or inability to understand or speak. Postictal symptoms include: amnesia for events, confusion, lethargy, sleepiness, headaches and muscle aches, transient focal weakness (Todd's paresis), and/or nausea or vomiting.

As one example of a therapeutic treatment, a composition disclosed herein can be administered to a subject who has Lennox-Gastaut Syndrome. An effective therapeutic treatment of Lennox-Gastaut Syndrome occurs when the types (tonic, atonic, atypical absence, myoclonic), severity, frequency, and/or duration of seizures is reduced or eliminated as measured by a standard subjective or objective seizure assessment, as described herein. As a further example, an effective therapeutic treatment of Lennox-Gastaut Syndrome occurs when cognitive dysfunction is reduced or eliminated as measured by a standard subjective or objective cognitive assessment. As another example, an effective therapeutic treatment of Lennox-Gastaut Syndrome occurs when the brain wave pattern of a subject suffering from Lennox-Gastaut Syndrome no longer exhibits the low [1.5- to 2.5-Hz] spike-and-wave pattern as measured by electroencephalogram (EEG).

As one example of a therapeutic treatment, a composition disclosed herein can be administered to a subject who has fragile X Syndrome. An effective therapeutic treatment of fragile X Syndrome occurs when symptoms such as: cognitive and intellectual problems including learning disabilities and abnormal speech; behavioral problems including hyperactivity, attention difficulties, avoidance of eye contact, and violent outbursts; and an unusual sensitivity to environmental stimuli are reduced or eliminated as measured by standard subjective or objective cognitive and/or behavioral assessments.

As one example of a therapeutic treatment, a composition disclosed herein can be administered to a subject who has dystonia. The dystonia can be idiopathic (not having a clear cause), genetic, or acquired (from environmental or other damage to the brain, or from exposure to certain types of medications). An effective therapeutic treatment of dystonia occurs when involuntary muscle contractions that cause slow repetitive movements or abnormal postures are reduced or eliminated as measured by standard subjective or objective muscle activity and/or brain activity assessments.

As one example of a therapeutic treatment, a composition disclosed herein can be administered to a subject who has Rett Syndrome. An effective therapeutic treatment of Rett Syndrome occurs when symptoms such as slowed growth, loss of normal movement and coordination, loss of communication abilities, abnormal hand movements, unusual eye movements, breathing problems, irritability and crying, cognitive disabilities, seizures, scoliosis, irregular heartbeat, and sleep disturbances are reduced or eliminated as measured by standard subjective or objective cognitive and/or behavioral assessments. In particular embodiments, an effective therapeutic treatment of Rett Syndrome occurs when the severity, frequency, or duration of seizures diminishes or disappears as measured by EEG. In particular embodiments, an effective therapeutic treatment of Rett Syndrome occurs when a subject suffering from Rett Syndrome maintains a stage or reverts to a less severe stage of Rett Syndrome, typically divided into four stages: Stage I (early onset); Stage II (rapid deterioration); Stage Ill (plateau); and Stage IV (late motor deterioration).

As one example of a therapeutic treatment, a composition disclosed herein can be administered to a subject who has Tourette Syndrome. Tourette Syndrome is characterized by sudden, brief, involuntary or semi-voluntary movements (motor tics) or sounds (vocal tics). Motor tics include eye blinks, head jerks, shoulder shrugs, facial or body contortions, touching, sniffing, jumping, and gesturing. Vocal tics include grunting, barking, yelping, throat clearing, and repeating words. In particular embodiments, an effective therapeutic treatment of Tourette Syndrome occurs when the amount and/or frequency of motor tics and/or vocal tics is reduced or eliminated. In particular embodiments, an effective therapeutic treatment of Tourette Syndrome occurs when the occurrence of motor tics and/or vocal tics are reduced to less than five or less than one a year.

As one example of a therapeutic treatment, a composition disclosed herein can be administered to a subject who has sleep apnea. An effective therapeutic treatment of sleep apnea occurs when symptoms such as snoring; choking, snorting, or gasping during sleep; pauses in breathing during sleep; waking up at night short of breath; and/or daytime sleepiness and fatigue are reduced in frequency or eliminated as measured by, for example, a recording of the subject's sleep or by a sleep partner.

As one example of a therapeutic treatment, a composition disclosed herein can be administered to a subject who has alcohol or opioid withdrawal syndrome. An effective therapeutic treatment of alcohol withdrawal syndrome occurs when onset and/or duration of symptoms of alcohol withdrawal syndrome such as: anxiety; excess perspiration; tremors, particularly in hands; dehydration; increased heart rate and blood pressure; insomnia; nausea and vomiting; diarrhea; seizures; hallucinations; delirium; extreme fluctuations in body temperature and blood pressure; and/or extreme agitation are reduced or eliminated as measured by an assessment tool such as Alcohol Withdrawal Scale (AWS; Nowak H, editor. Nursing education and nursing management of alcohol and other drugs. Sydney: CEIDA; 1989). In particular embodiments, an effective therapeutic treatment of opioid withdrawal syndrome occurs when onset and/or duration of symptoms of opioid withdrawal syndrome such as: nausea and vomiting; anxiety; insomnia; hot and cold flushes; perspiration; muscle cramps; watery discharge from eyes and nose; and/or diarrhea are reduced or eliminated as measured by an assessment tool such as Short Opioid Withdrawal Scale (SOWS; Gossop M. The development of a short opiate withdrawal scale. Addictive Behaviors. 1990; 15: 487-490).

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more active agent(s) can be administered simultaneously or within a selected time window, such as within 10 minutes, 1 hour, 3 hour, 10 hour, 15 hour, 24 hour, or 48 hour time windows or when the complementary active agent(s) is within a clinically-relevant therapeutic window.

One or more active agent(s) can be administered orally, nasally, or by inhalation. Nasal administration and administration by inhalation includes absorption of the active ingredient through nasal mucous membranes or lung tissue, respectively, for entry into the circulatory system of a subject for systemic effects. In particular embodiments, oral administration includes providing an oral dosage form that can be swallowed and the active ingredient absorbed through the gastrointestinal tract. In particular embodiments, oral administration includes providing an oral dosage form that can be absorbed through the mucous membranes of the oral cavity for entry into the circulatory system of a subject for systemic effects. Oral transmucosal delivery of a stabilized cannabinoid includes delivery across any tissue of the mouth, pharynx, larynx, trachea, or upper gastrointestinal tract, including the sublingual, gingival and palatal mucosal tissues.

In particular embodiments, the administration of the present compositions may include 1, 2, 3, 4, 5, 6, 7 or 8 inhalations of the present compositions in each nostril one, two, three, four or five times a day. In particular embodiments, the administration of the present compositions may include only 1 inhalation in each nostril per day.

(viii) Exemplary Embodiments.

1. A solid dosage form including:
(a) (i) 15-30% 1,2-distearoyl-sn-glycero-3-phosphocholine (DSPC) or 15-30% 1,2-dipalmitoyl-sn-glycero-3-phosphocholine (DPPC) and (ii) 10-30% tetrahydrocannabinol (THC); or
(b) (i) 5-30% DSPC or 15-30% DPPC and (ii) 10-50% cannabidiol (CBD), wherein the solid dosage form is an inhalable dry powder, and wherein the solid dosage form retains at least 90% of the THC or CBD through at least 32 weeks following formulation.

2. The solid dosage form of embodiment 1, wherein the inhalable dry powder includes fumaryl diketopiperazine having the following structure:

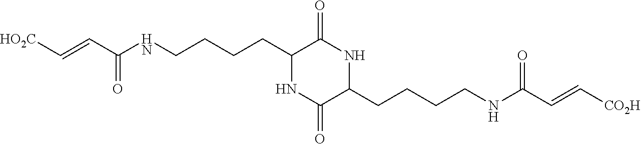 [SMILES: O=C(O)/C=C/C(=O)NCCCCC1NC(=O)C(CCCCNC(=O)/C=C/C(=O)O)NC1=O]

3. The solid dosage form of embodiment 1 or 2, wherein the inhalable dry powder further includes amino acids, mannitol, and/or lactose.

4. The solid dosage form of embodiment 3, wherein the amino acids include leucine or isoleucine.

5. The solid dosage form of any of embodiments 1-4, wherein the inhalable dry powder includes particles having an aerodynamic diameter of between 0.5 and 10 μm.

6. A solid dosage form including a cannabinoid directly stabilized by a phospholipid.

7. The solid dosage form of embodiment 6, wherein the phospholipid includes a phosphatidylcholine (PC).

8. The solid dosage form of embodiment 7, wherein the PC includes 1,2-distearoyl-sn-glycero-3-phosphocholine (DSPC) or 1,2-dipalmitoyl-sn-glycero-3-phosphocholine (DPPC).

9. The solid dosage form of embodiment 7 or 8, including 5-30% w/w PC.

10. The solid dosage form of any of embodiments 7-9, including 5-20% w/w PC.

11. The solid dosage form of any of embodiments 6-10, including 0.5%-50% w/w cannabinoid.

12. The solid dosage form of any of embodiments 6-11, including 0.5-30% w/w cannabinoid.

13. The solid dosage form of any of embodiments 6-12, including 0.5%-50% w/w tetrahydrocannabinol (THC).

14. The solid dosage form of any of embodiments 6-13, including 0.5%-50% w/w cannabidiol (CBD).

15. The solid dosage form of any of embodiments 8-14, including 5-20% w/w DSPC or 5-20% w/w DPPC and 10-30% w/w THC or 10-50% w/w CBD wherein the solid dosage form retains at least 90% of the THC or CBD through at least 32 weeks following formulation.

16. The solid dosage form of any of embodiments 8-15, including 15% w/w DSPC and 25% w/w THC wherein the solid dosage form retains at least 90% of the THC through at least 32 weeks following formulation.

17. The solid dosage form of any of embodiments 8-15, including 5% w/w DSPC and 25% w/w CBD wherein the solid dosage form retains at least 90% of the CBD through at least 24 weeks following formulation.

18. The solid dosage form of any of embodiments 8-15, including 5-20% w/w DSPC or 10-20% w/w DPPC and 10-50% w/w CBD wherein the solid dosage form retains at least 90% of the CBD through at least 63 weeks following formulation.

19. The solid dosage form of any of embodiments 6-18, wherein the cannabinoid includes THC, CBD, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA) and/or mixtures thereof.

20. The solid dosage form of any of embodiments 6-19, wherein the cannabinoid includes a synthetic cannabinoid.

21. The solid dosage form of any of embodiments 6-20, wherein the dosage form is a dry powder.

22. The solid dosage form of embodiment 21, wherein the dry powder is formulated for nasal administration.

23. The solid dosage form of embodiment 21 or 22, wherein the dry powder is an inhalable dry powder.

24. The solid dosage form of embodiment 23, wherein the inhalable dry powder includes 0.5-50% w/w THC.

25. The solid dosage form of embodiment 23 or 24, wherein the inhalable powder includes 0.5-50% w/w CBD.

26. The solid dosage form of any of embodiments 23-25, wherein the inhalable dry powder includes fumaryl diketopiperazine (FDKP) having the following structure:

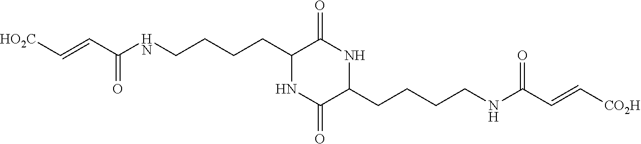 [SMILES: O=C(O)/C=C/C(=O)NCCCCC1NC(=O)C(CCCCNC(=O)/C=C/C(=O)O)NC1=O]

27. The solid dosage form of any of embodiments 23-26, wherein the inhalable dry powder further includes amino acids, mannitol, and/or lactose.

28. The solid dosage form of embodiment 27, wherein the amino acids include leucine or isoleucine.

29. The solid dosage form of any of embodiments 23-28, wherein the inhalable dry powder includes particles having an aerodynamic diameter of between 0.5 and 10 μm.

30. The solid dosage form of embodiment 21, wherein the dry powder is formulated into an oral composition.

31. The solid dosage form of embodiment 30, wherein the oral composition includes 0.5-50% w/w THC.

32. The solid dosage form of embodiment 30 or 31, wherein the oral composition includes 0.5-50% w/w CBD.

33. The solid dosage form of any of embodiments 30-32, wherein the oral composition is a tablet or capsule.

34. The solid dosage form of any of embodiments 30-33, wherein the oral composition is formulated for oral transmucosal delivery.

35. The solid dosage form of any of embodiments 30-34, wherein the oral composition includes an N-acylated fatty amino acid.

36. The solid dosage form of embodiment 35, wherein the N-acylated fatty amino acid includes one or more of Compounds XVII-LI (FIG. 11) or Compounds a-r (FIG. 12).

37. The solid dosage form of embodiment 35 or 36, wherein the N-acylated fatty amino acid has the formula:

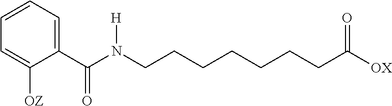

wherein X and Z are independently hydrogen, a monovalent cation, a divalent metal cation, or an organic cation.

38. The solid dosage form of embodiment 37, wherein the monovalent cation is sodium, potassium, or ammonium.

39. The solid dosage form of embodiment 37 or 38, wherein the divalent metal cation is calcium or magnesium.

40. The solid dosage form of any of embodiments 37-39, wherein the organic cation is tetramethylammonium.

41. The solid dosage form of any of embodiments 37-40, wherein X is hydrogen.

42. The solid dosage form of any of embodiments 37-40, wherein X is sodium, potassium, or ammonium.

43. The solid dosage form of any of embodiments 37-40, wherein X is calcium or magnesium.

44. The solid dosage form of any of embodiments 37-40, wherein X is tetramethylammonium.

45. The solid dosage form of any of embodiments 37-45, wherein Z is hydrogen.

46. The solid dosage form of any of embodiments 37-44, wherein Z is sodium or potassium.

47. The solid dosage form of any of embodiments 37-44, wherein Z is calcium or magnesium.

48. The solid dosage form of any of embodiments 37-41, and 45, wherein X is hydrogen and Z is hydrogen.

49. The solid dosage form of any of embodiments 37-40, 42, and 45, wherein X is sodium and Z is hydrogen.

50. The solid dosage form of any of embodiments 37-41, and 46, wherein X is hydrogen and Z is sodium.

51. The solid dosage form of embodiments 37-40, 42, and 46, wherein X is sodium and Z is sodium.

52. A method of forming a solid dosage form with a directly stabilized cannabinoid including:
adding a cannabinoid and a phospholipid to a receptacle at a ratio to form a cannabinoid/phospholipid solution, wherein the cannabinoid is within a solvent; and
removing the solvent,
thereby forming the solid dosage form with the directly stabilized cannabinoid.

53. The method of embodiment 52, wherein the phospholipid includes a phosphatidylcholine (PC).

54. The method of embodiment 52 or 53, wherein the ratio comprises a w/w ratio of cannabinoid to phospholipid of 1:1, 1:2, 1:3, 1:4, 1:5, 1:6, 5:1, 4:1, 3:1, or 2:1.

55. The method of any of embodiments 52-54, wherein the PC includes 1,2-distearoyl-sn-glycero-3-phosphocholine (DSPC) or 1,2-dipalmitoyl-sn-glycero-3-phosphocholine (DPPC).

56. The method of any of embodiments 52-55, wherein the cannabinoid includes tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA) and/or mixtures thereof.

57. The method of any of embodiments 52-56, wherein the cannabinoid includes a synthetic cannabinoid.

58. The method of any of embodiments 52-57, further including mixing the cannabinoid with the solvent prior to the adding.

59. The method of embodiment 58, wherein the solvent is ethanol, methanol, acetone, tetrahydrofuran, or ethyl acetate.

60. The method of embodiment 58 or 59, wherein the solvent includes ethanol and water, methanol and water, acetone and water, or tetrahydrofuran and water.

61. The method of any of embodiments 52-60, wherein the removing includes spray-drying or drying in a vacuum oven.

62. The method of any of embodiments 52-61, wherein the removing results in a dry powder solid dosage form.

63. The method of embodiment 62, wherein the dry powder solid dosage form has 5-30% w/w PC.

64. The method of embodiment 62 or 63, wherein the dry powder solid dosage form has 5-20% w/w PC.

65. The method of any of embodiments 62-64, wherein the dry powder solid dosage form has 0.5-50% w/w cannabinoid.

66. The method of any of embodiments 62-65, wherein the dry powder solid dosage form has 0.5-30% w/w cannabinoid.

67. The method of any of embodiments 62-66, wherein the dry powder solid dosage form has 0.5%-50% w/w THC.

68. The method of any of embodiments 62-67, wherein the dry powder solid dosage form has 0.5%-50% w/w CBD.

69. The method of any of embodiments 62-68, wherein the dry powder solid dosage form has 5-20% w/w DSPC or DPPC and 10-50% w/w THC or CBD.

70. The method of any of embodiments 62-69, wherein the dry powder solid dosage form has 15% w/w DSPC and 25% w/w THC.

71. The method of any of embodiments 62-69, wherein the dry powder solid dosage form has 5% w/w DSPC and 25% CBD.

72. The method of any of embodiments 52-71, further including adding fumaryl diketopiperazine (FDKP) having the following structure:

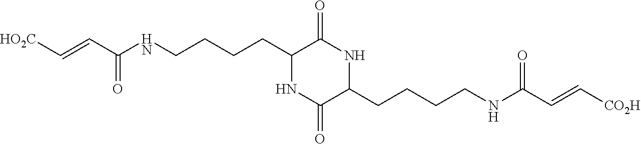 [SMILES: O=C(O)/C=C/C(=O)NCCCCC1NC(=O)C(CCCCNC(=O)/C=C/C(=O)O)NC1=O]

to a solvent to form an excipient suspension; and
combining the excipient suspension with the cannabinoid/phospholipid solution prior to the removing.

73. The method of embodiment 72, further including adding amino acids, mannitol, and/or lactose to the excipient suspension.

74. The method of embodiment 73, wherein the amino acids include leucine or isoleucine.

75. The method of any of embodiments 72-74, wherein the removing results in an inhalable dry powder solid dosage form.

76. The method of any of embodiments 52-75, wherein the solvent including the cannabinoid and the solvent of the excipient suspension are the same.

77. The method of any of embodiments 52-76, wherein the solvent is ethanol.

78. The method of any of embodiments 75-77, further including loading the inhalable dry powder solid dosage form into an inhalable cartridge.

79. The method of embodiment 78, wherein the loaded inhalable dry powder solid dosage form has 0.5-50% w/w THC.

80. The method of embodiment 78 or 79, wherein the loaded inhalable dry powder solid dosage form has 0.5-50% w/w CBD.

81. The method of any of embodiments 75-80, wherein the inhalable dry powder solid dosage includes particles having an aerodynamic diameter of between 0.5 and 5.8 μm.

82. The method of any of embodiments 52-71, further including formulating the dry powder for nasal administration.

83. The method of any of embodiments 52-71, further including formulating the dry powder for oral delivery.

84. The method of any of embodiments 52-71 and 83, further including formulating the dry powder for oral transmucosal delivery.

85. The method of any of embodiments 52-71, 83, and 84, further including loading the dry powder solid dosage form into capsules.

86. The method of any of embodiments 52-71, 83, and 84, further including pressing the dry powder solid dosage form into tablets.

87. The method of any of embodiments 52-71, and 83-86, further including adding an N-acylated fatty amino acid to the solvent.

88. The method of embodiment 87, wherein the N-acylated fatty amino acid includes one or more of Compounds XVII-LI (FIG. 11) or Compounds a-r (FIG. 12).

89. The method of embodiment 87 or 88, wherein the N-acylated fatty amino acid has the formula:

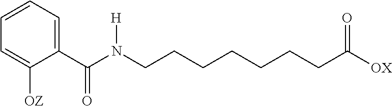

wherein X and Z are independently hydrogen, a monovalent cation, a divalent metal cation, or an organic cation.

90. The method of embodiment 89, wherein the monovalent cation is sodium, potassium, or ammonium.

91. The method of embodiment 89 or 90, wherein the divalent metal cation is calcium or magnesium.

92. The method of any of embodiments 89-91, wherein the organic cation is tetramethylammonium.

93. The method of any of embodiments 89-92, wherein X is hydrogen.

94. The method of any of embodiments 89-92, wherein X is sodium, potassium, or ammonium.

95. The method of any of embodiments 89-92, wherein X is calcium or magnesium.

96. The method of any of embodiments 89-92, wherein X is tetramethylammonium.

97. The method of any of embodiments 89-96, wherein Z is hydrogen.

98. The method of any of embodiments 89-96, wherein Z is sodium or potassium.

99. The method of any of embodiments 89-96, wherein Z is calcium or magnesium.

100. The method of any of embodiments 89-93, and 97, wherein X is hydrogen and Z is hydrogen.

101. The method of any of embodiments 89-92, 94, and 97, wherein X is sodium and Z is hydrogen.

102. The method of any of embodiments 89-93, and 98, wherein X is hydrogen and Z is sodium.

103. The method of any of embodiments 89-92, 94, and 98, wherein X is sodium and Z is sodium.

(ix) Experimental Examples.

Example 1. THC inhalation powders were prepared using a Buchi B-290 spray drier. Batches were prepared with identical processing conditions (e.g., THC content, THC lot), but with varying phosphatidylcholines (DSPC or DPPC) and phosphatidylcholine content (Table 1). Batches were placed into tightly closed glass vials, which were stored in an amber nitrogen-purged desiccator cabinet at uncontrolled ambient temperature. Samples were removed at various time points for analysis. To analyze stability trends as a function of phosphatidylcholine type and content, data were extrapolated (linear) through the latest timepoint tested for the group being compared.

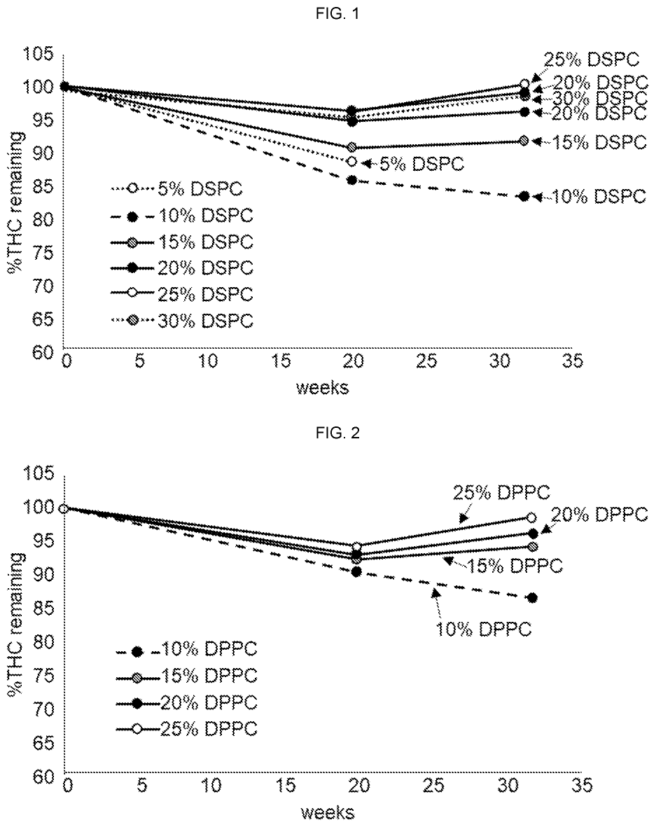

Stability data through 32 weeks demonstrated that increasing DSPC or DPPC (FIG. 1) increased THC stability within the inhalation powder. Trends observed in the data include:
Powders containing 15% or more phosphatidylcholine (either DSPC or DPPC) retained >90% THC assay through the 32-week time point;
Powders with >20% phosphatidylcholine (either DSPC or DPPC) had similar stability, retaining >95% THC assay through the 32-week time point.

TABLE 1
Stability of THC within inhalation powders including
varying phosphatidylcholines (DSPC and DPPC)
and phosphatidylcholine contents.

| | DSPC | DPPC | % THC remaining | | | |
|---|---|---|---|---|---|---|
| Lot | content | content | | 20 | 32 | 36 |
| number | (%) | (%) | T0 | weeks | weeks | weeks |
| R0007.30 | 5 | — | 100 | 88.6 | — | — |
| R0007.37 | 10 | — | 100 | 85.7 | 83.3 | — |
| R0007.44 | 10 | — | 100 | — | — | 83.3 |
| R0007.31 | 15 | — | 100 | 90.7 | 91.7 | — |
| R0007.32 | 20 | — | 100 | 94.8 | 96.2 | — |
| R0007.61 | 20 | — | 100 | 96.3 | 99.1 | — |
| R0007.45 | 25 | — | 100 | 96.2 | 100.5 | — |
| R0007.46 | 30 | — | 100 | 95.2 | 98.7 | — |
| R0007.36 | — | 10 | 100 | 90.4 | 86.6 | — |
| R0007.34 | — | 15 | 100 | 92.5 | 94.4 | — |
| R0007.35 | — | 20 | 100 | 93.2 | 96.3 | — |
| R0007.47 | — | 25 | 100 | 94.3 | 98.7 | — |

For all batches, THC content was 26%, feed rate was 10%, and ethanol content was 90%.

In a follow-up study, THC inhalation powders were prepared using a Buchi B-290 spray drier. Batches were prepared with varying THC content (10-30%) and varying DSPC content (0-20%). A single lot of THC extract was used to prepare all inhalation powders. Batches were placed into tightly closed glass vials, which were stored at 25° C./60% RH (relative humidity) for up to 36 weeks. A subset of powders was placed into glass vials, purged with nitrogen before closing tightly, and then stored at 25° C./60% RH for up to 36 weeks (FIG. 6).

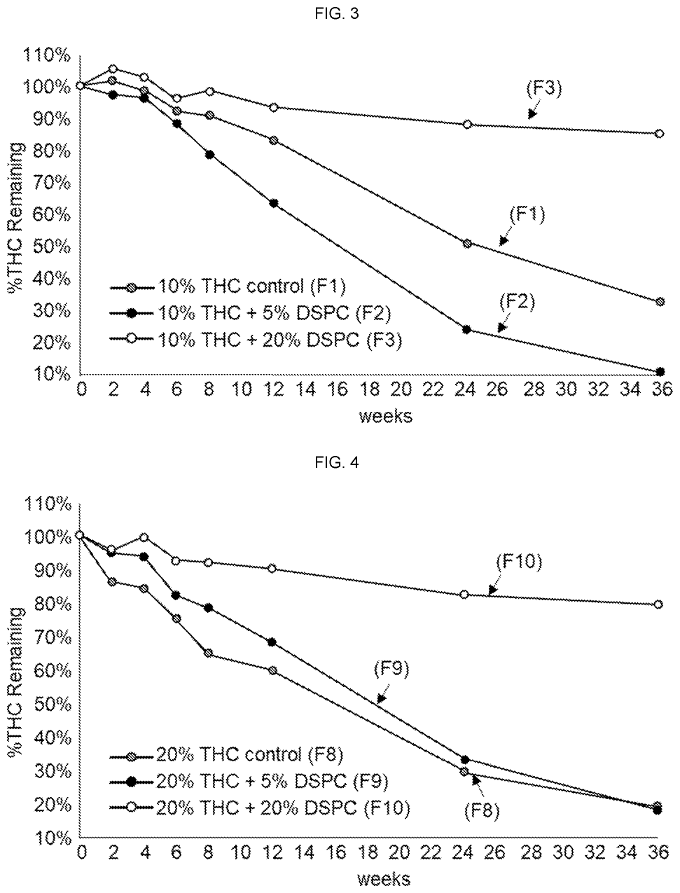

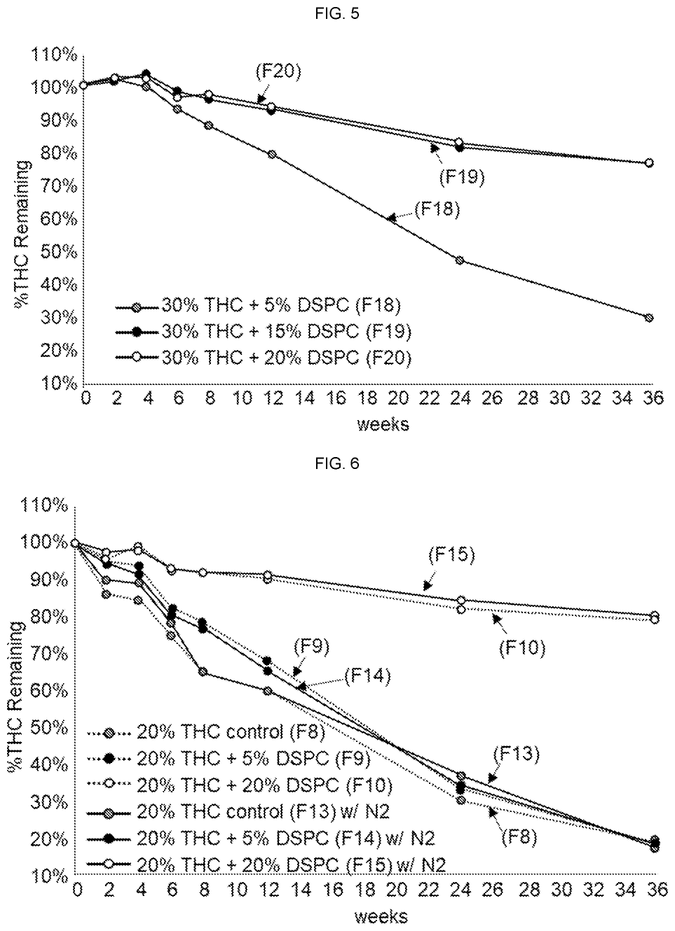

Stability data through 36 weeks were in line with results from the initial stability study and demonstrated that increasing DSPC increased THC stability within the inhalation powder. FIG. 3 shows the effect of DSPC content on stability of 10% THC inhalation powders; FIG. 4 shows the effect of DSPC content on stability of 20% THC inhalation powders; FIG. 5 shows the effect of DSPC content on stability of 30% THC inhalation powders; and FIG. 6 shows the effect of a nitrogen atmosphere on stability of 20% THC inhalation powders.

Some trends observed in the data include:
Powders with 0-5% DSPC had similar, poor stability;
Powders containing 15-20% DSPC had similar stability;
When DSPC content was fixed at a relatively high content (e.g., 20%), powder stability at the 36-week time point improved as THC content decreased. Specifically, the 10%, 20% and 30% powders had 85%, 79%, and 76% THC remaining, respectively, at the 36-week time point;
When DSPC content was fixed at a relatively low content (e.g., 5%), powder stability at the 36-week time point improved as THC content increased. Specifically, the 10%, 20% and 30% powders had 11%, 20%, and 30% THC remaining, respectively, at the 36-week time point; and
There was no difference in stability between samples exposed to air or those stored in an inert (nitrogen) atmosphere (FIG. 6).

Example 2. Analysis of CBD inhalation powders after storage at uncontrolled ambient conditions for up to 63 weeks demonstrated that inclusion of >5% DSPC or >15% DPPC (lowest amount tested) improved stability compared to control powders without phosphatidylcholines (i.e. DSPC and DPPC).

Example 1 showed that the stability of THC inhalation powders improves with the presence of 15% of the phosphatidylcholine DSPC (1,2-distearoyl-sn-glycero-3-phosphocholine) or DPPC (dipalmitoylphosphatidylcholine). To determine whether CBD is stabilized in a similar manner, 26% CBD inhalation powders, containing 5-20% DSPC, 15-20% DPPC, or no phosphatidylcholine (as a control), were prepared using a Buchi B-290 spray drier. After preparation, powders were stored in tightly closed amber glass vials at uncontrolled ambient temperature. Samples were removed at various timepoints for analysis.

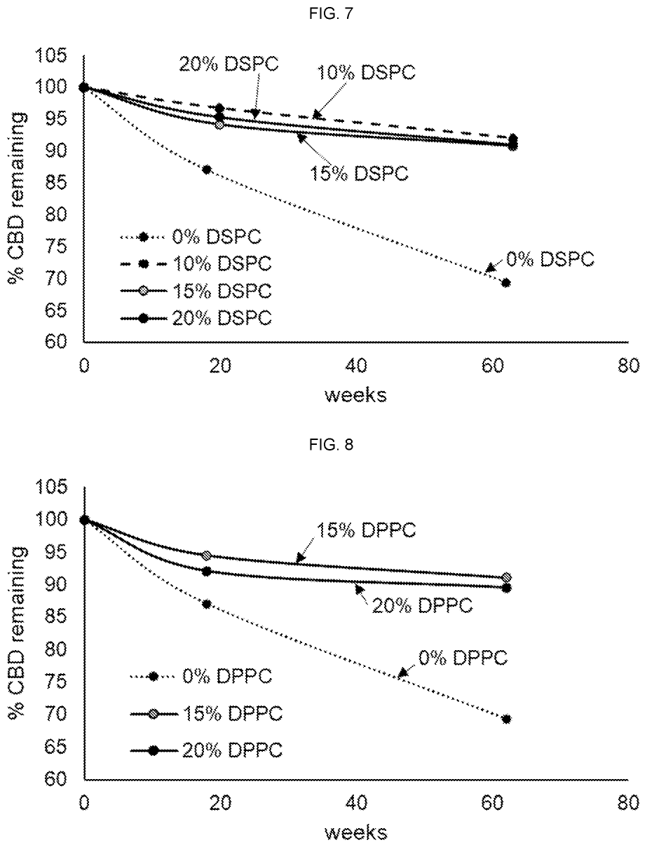

Following storage at uncontrolled ambient conditions for over a year, a 26% CBD powder without phosphatidylcholine had 69.4% CBD remaining (Table 2). Powders with DSPC or DPPC retained >90% assay following storage at the same conditions and for the same duration. There were no apparent differences in stability when comparing the phosphatidylcholine used (DSPC or DPPC) or the amount of the phosphatidylcholine included (Table 2, FIG. 7 and FIG. 8).

TABLE 2
Stability of CBD inhalation powders made with varying
phosphatidylcholine and phosphatidylcholine content.

| | | % CBD remaining | | | | |
|---|---|---|---|---|---|---|
| Lot | phosphatidylcholine | | 18 | 20 | 62 | 63 |
| number | content, type | T0 | weeks | weeks | weeks | weeks |
| R00011.083 | 10% DSPC | 100 | — | 96.7 | — | 92.0 |
| R00011.085 | 15% DSPC | 100 | — | 94.2 | — | 90.8 |
| R00011.086 | 20% DSPC | 100 | — | 95.3 | — | 91.0 |
| R00011.087 | none | 100 | 87.1 | — | 69.4 | — |
| R00011.090 | 15% DPPC | 100 | 94.5 | — | 91.1 | — |
| R00011.091 | 20% DPPC | 100 | 92.1 | — | 89.6 | — |

Stability of follow-up CBD powders. Three additional 26% CBD inhalation powders were prepared to determine the impact of DSPC content between 0 and 10% and to confirm the previous results.

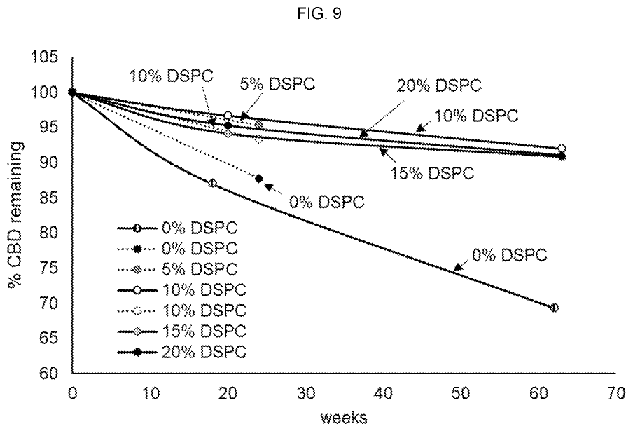

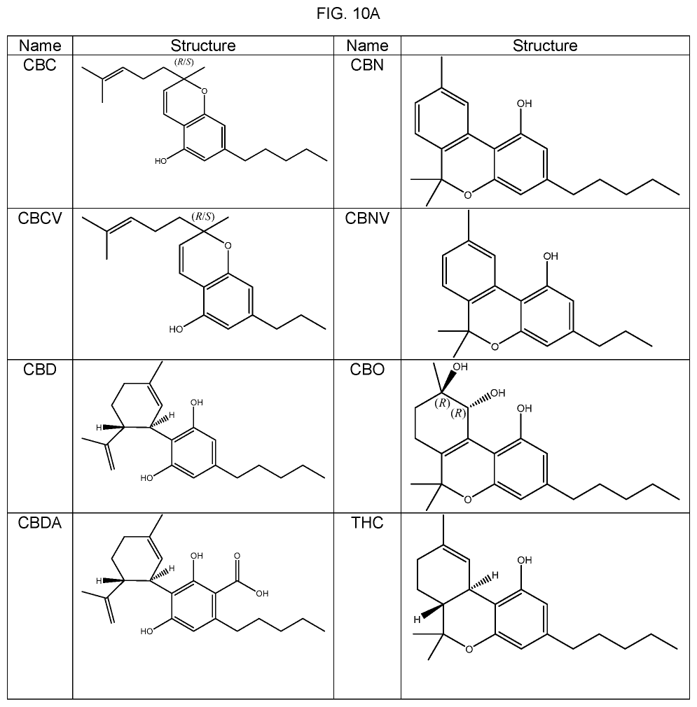

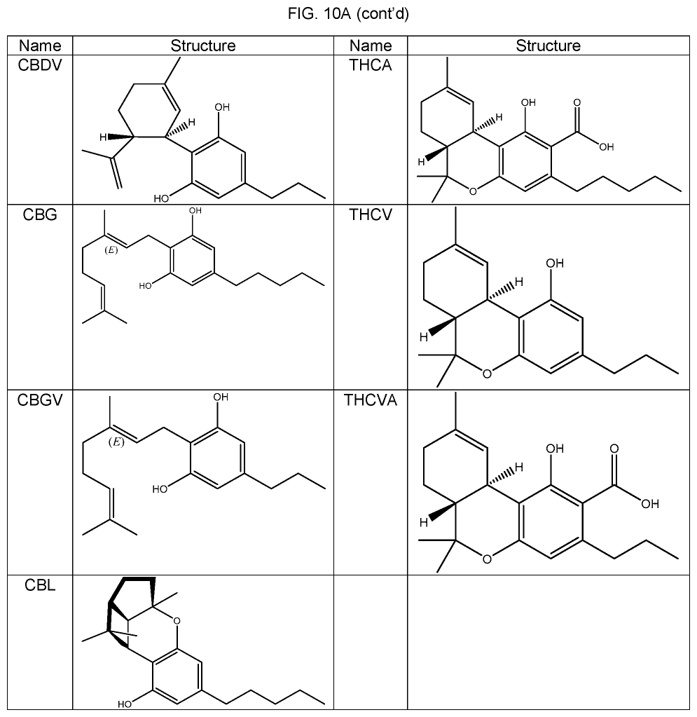

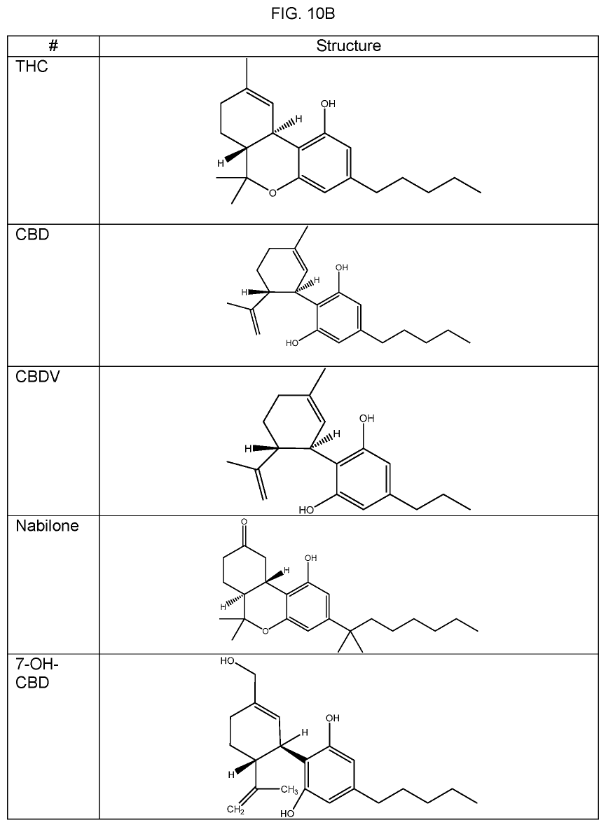

After 24 weeks of storage at uncontrolled ambient conditions, the 26% CBD powder without phosphatidylcholine had 87.7% CBD remaining (Table 3), while the powders with 5% and 10% DSPC retained 95.4% and 93.4% assay, respectively. Stability results for the follow up powders (FIG. 9, dotted lines) were in line with the data from the original powders (FIG. 9, solid lines).

TABLE 3
Stability of CBD inhalation powders made with 0-10% DSPC.

| | |
|---|---|
| DSPC | % CBD remaining |

| Lot number | content (%) | T0 | 24 weeks |
|---|---|---|---|
| R00011.120 | 0 | 100 | 87.7 |
| R00011.121 | 5 | 100 | 95.4 |
| R00011.122 | 10 | 100 | 93.4 |

(x) Closing Paragraphs. As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to directly stabilize a cannabinoid with a phospholipid in a solid dosage form.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A solid dosage form comprising:
(a) (i) 15-30% 1,2-distearoyl-sn-glycero-3-phosphocholine (DSPC) or 15-30% 1,2-dipalmitoyl-sn-glycero-3-phosphocholine (DPPC) and (ii) 10-30% tetrahydrocannabinol (THC); or
(b) (i) 5-30% DSPC or 15-30% DPPC and (ii) 10-50% cannabidiol (CBD),
wherein the solid dosage form is an inhalable dry powder, and wherein the solid dosage form retains at least 90% of the THC or CBD through at least 32 weeks following formulation.

2. The solid dosage form of claim 1, wherein the inhalable dry powder comprises fumaryl diketopiperazine having the following structure:

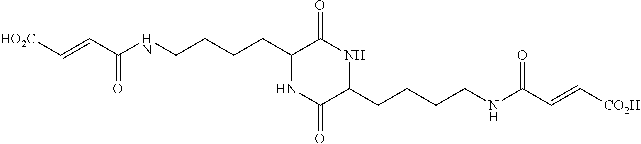 [SMILES: O=C(O)/C=C/C(=O)NCCCCC1NC(=O)C(CCCCNC(=O)/C=C/C(=O)O)NC1=O]

3. The solid dosage form of claim 1, wherein the inhalable dry powder further comprises amino acids, mannitol, and/or lactose.

4. The solid dosage form of claim 3, wherein the amino acids comprise leucine or isoleucine.

5. The solid dosage form of claim 1, wherein the inhalable dry powder comprises particles having an aerodynamic diameter of between 0.5 and 10 μm.

6. A solid dosage form comprising a cannabinoid directly stabilized by a phospholipid.

7. The solid dosage form of claim 6, wherein the phospholipid comprises a phosphatidylcholine (PC).

8. The solid dosage form of claim 7, wherein the PC comprises 1,2-distearoyl-sn-glycero-3-phosphocholine (DSPC) or 1,2-dipalmitoyl-sn-glycero-3-phosphocholine (DPPC).

9. The solid dosage form of claim 7, comprising 5-30% w/w PC.

10. The solid dosage form of claim 7, comprising 5-20% w/w PC.

11. The solid dosage form of claim 6, comprising 0.5%-50% w/w cannabinoid.

12. The solid dosage form of claim 6, comprising 0.5-30% w/w cannabinoid.

13. The solid dosage form of claim 6, comprising 0.5%-50% w/w tetrahydrocannabinol (THC).

14. The solid dosage form of claim 6, comprising 0.5%-50% w/w cannabidiol (CBD).

15. The solid dosage form of claim 8, comprising 5-20% w/w DSPC or 5-20% w/w DPPC and 10-30% w/w THC or 10-50% w/w CBD wherein the solid dosage form retains at least 90% of the THC or CBD through at least 32 weeks following formulation.

16. The solid dosage form of claim 8, comprising 15% w/w DSPC and 25% w/w THC wherein the solid dosage form retains at least 90% of the THC through at least 32 weeks following formulation.

17. The solid dosage form of claim 8, comprising 5% w/w DSPC and 25% w/w CBD wherein the solid dosage form retains at least 90% of the CBD through at least 24 weeks following formulation.

18. The solid dosage form of claim 8, comprising 5-20% w/w DSPC or 10-20% w/w DPPC and 10-50% w/w CBD wherein the solid dosage form retains at least 90% of the CBD through at least 63 weeks following formulation.

19. The solid dosage form of claim 6, wherein the cannabinoid comprises THC, CBD, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA) and/or mixtures thereof.

20. The solid dosage form of claim 6, wherein the cannabinoid comprises a synthetic cannabinoid.

21. The solid dosage form of claim 6, wherein the dosage form is a dry powder.

22. The solid dosage form of claim 21, wherein the dry powder is formulated for nasal administration.

23. The solid dosage form of claim 21, wherein the dry powder is an inhalable dry powder.

24. The solid dosage form of claim 23, wherein the inhalable dry powder comprises 0.5-50% w/w THC.

25. The solid dosage form of claim 23, wherein the inhalable powder comprises 0.5-50% w/w CBD.

26. The solid dosage form of claim 23, wherein the inhalable dry powder comprises fumaryl diketopiperazine (FDKP) having the following structure:

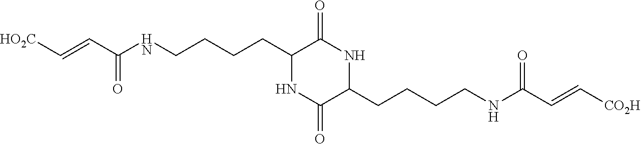 [SMILES: O=C(O)/C=C/C(=O)NCCCCC1NC(=O)C(CCCCNC(=O)/C=C/C(=O)O)NC1=O]

27. The solid dosage form of claim 23, wherein the inhalable dry powder further comprises amino acids, mannitol, and/or lactose.

28. The solid dosage form of claim 27, wherein the amino acids comprise leucine or isoleucine.

29. The solid dosage form of claim 23, wherein the inhalable dry powder comprises particles having an aerodynamic diameter of between 0.5 and 10 μm.

30. The solid dosage form of claim 21, wherein the dry powder is formulated into an oral composition.

31. The solid dosage form of claim 30, wherein the oral composition comprises 0.5-50% w/w THC.

32. The solid dosage form of claim 30, wherein the oral composition comprises 5-50% w/w CBD.

33. The solid dosage form of claim 30, wherein the oral composition is a tablet or capsule.

34. The solid dosage form of claim 30, wherein the oral composition is formulated for oral transmucosal delivery.

35. The solid dosage form of claim 30, wherein the oral composition comprises an N-acylated fatty amino acid.

36. The solid dosage form of claim 35, wherein the N-acylated fatty amino acid comprises one or more of Compounds XVII-LI (FIG. 11) or Compounds a-r (FIG. 12).

37. The solid dosage form of claim 35, wherein the N-acylated fatty amino acid has the formula: 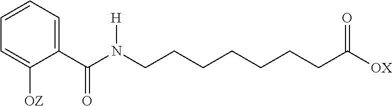
wherein X and Z are independently hydrogen, a monovalent cation, a divalent metal cation, or an organic cation.

38. The solid dosage form of claim 37, wherein the monovalent cation is sodium, potassium, or ammonium.

39. The solid dosage form of claim 37, wherein the divalent metal cation is calcium or magnesium.

40. The solid dosage form of claim 37, wherein the organic cation is tetramethylammonium.

41. The solid dosage form of claim 37, wherein X is hydrogen.

42. The solid dosage form of claim 37, wherein X is sodium, potassium, or ammonium.

43. The solid dosage form of claim 37, wherein X is calcium or magnesium.

44. The solid dosage form of claim 37, wherein X is tetramethylammonium.

45. The solid dosage form of claim 37, wherein Z is hydrogen.

46. The solid dosage form of claim 37, wherein Z is sodium or potassium.

47. The solid dosage form of claim 37, wherein Z is calcium or magnesium.

48. The solid dosage form of claim 37, wherein X is hydrogen and Z is hydrogen.

49. The solid dosage form of claim 37, wherein X is sodium and Z is hydrogen.

50. The solid dosage form of claim 37, wherein X is hydrogen and Z is sodium.

51. The solid dosage form of claim 37, wherein X is sodium and Z is sodium.

52. A method of forming a solid dosage form with a directly stabilized cannabinoid comprising:
adding a cannabinoid and a phospholipid to a receptacle at a ratio to form a cannabinoid/phospholipid solution, wherein the cannabinoid is within a solvent; and
removing the solvent,
thereby forming the solid dosage form with the directly stabilized cannabinoid.

53. The method of claim 52, wherein the phospholipid comprises a phosphatidylcholine (PC).

54. The method of claim 53, wherein the PC comprises 1,2-distearoyl-sn-glycero-3-phosphocholine (DSPC) or 1,2-dipalmitoyl-sn-glycero-3-phosphocholine (DPPC).

55. The method of claim 52, wherein the cannabinoid comprises tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA) and/or mixtures thereof.

56. The method of claim 52, wherein the cannabinoid comprises a synthetic cannabinoid.

57. The method of claim 52, further comprising mixing the cannabinoid with the solvent prior to the adding.

58. The method of claim 57, wherein the solvent is ethanol, methanol, acetone, tetrahydrofuran, or ethyl acetate.

59. The method of claim 57, wherein the solvent comprises ethanol and water, methanol and water, acetone and water, or tetrahydrofuran and water.

60. The method of claim 52, wherein the removing comprises spray-drying or drying in a vacuum oven.

61. The method of claim 52, wherein the removing results in a dry powder solid dosage form.

62. The method of claim 61, wherein the dry powder solid dosage form has 5-30% w/w PC.

63. The method of claim 61, wherein the dry powder solid dosage form has 5-20% w/w PC.

64. The method of claim 61, wherein the dry powder solid dosage form has 0.5-50% w/w cannabinoid.

65. The method of claim 61, wherein the dry powder solid dosage form has 0.5-30% w/w cannabinoid.

66. The method of claim 61, wherein the dry powder solid dosage form has 0.5%-50% w/w THC.

67. The method of claim 61, wherein the dry powder solid dosage form has 0.5%-50% w/w CBD.

68. The method of claim 61, wherein the dry powder solid dosage form has 5-20% w/w DSPC or DPPC and 10-50% w/w THC or CBD.

69. The method of claim 61, wherein the dry powder solid dosage form has 15% w/w DSPC and 25% w/w THC.

70. The method of claim 61, wherein the dry powder solid dosage form has 5% w/w DSPC and 25% CBD.

71. The method of claim 52, further comprising adding fumaryl diketopiperazine (FDKP) having the following structure: 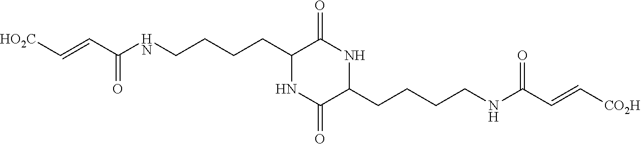 [SMILES: O=C(O)/C=C/C(=O)NCCCCC1NC(=O)C(CCCCNC(=O)/C=C/C(=O)O)NC1=O]
to a solvent to form an excipient suspension; and
combining the excipient suspension with the cannabinoid/phospholipid solution prior to the removing.

72. The method of claim 71, further comprising adding amino acids, mannitol, and/or lactose to the excipient suspension.

73. The method of claim 72, wherein the amino acids comprise leucine or isoleucine.

74. The method of claim 71, wherein the removing results in an inhalable dry powder solid dosage form.

75. The method of claim 71, wherein the solvent comprising the cannabinoid and the solvent of the excipient suspension are the same.

76. The method of claim 75, wherein the solvent is ethanol.

77. The method of claim 74, further comprising loading the inhalable dry powder solid dosage form into an inhalable cartridge.

78. The method of claim 77, wherein the loaded inhalable dry powder solid dosage form has 0.5-50% w/w THC.

79. The method of claim 77, wherein the loaded inhalable dry powder solid dosage form has 0.5-50% w/w CBD.

80. The method of claim 74, wherein the inhalable dry powder solid dosage comprises particles having an aerodynamic diameter of between 0.5 and 5.8 μm.

81. The method of claim 61, further comprising formulating the dry powder for nasal administration.

82. The method of claim 61, further formulating the dry powder for oral delivery.

83. The method of claim 61, further comprising formulating the dry powder for oral transmucosal delivery.

84. The method of claim 61, further comprising loading the dry powder solid dosage form into capsules.

85. The method of claim 61, further comprising pressing the dry powder solid dosage form into tablets.

86. The method of claim 52, further comprising adding an N-acylated fatty amino acid to the solvent.

87. The method of claim 85, wherein the N-acylated fatty amino acid comprises one or more of Compounds XVII-LI (FIG. 11) or Compounds a-r (FIG. 12).

88. The method of claim 85, wherein the N-acylated fatty amino acid has the formula: 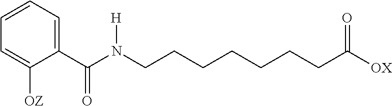
wherein X and Z are independently hydrogen, a monovalent cation, a divalent metal cation, or an organic cation.

89. The method of claim 87, wherein the monovalent cation is sodium, potassium, or ammonium.

90. The method of claim 87, wherein the divalent metal cation is calcium or magnesium.

91. The method of claim 87, wherein the organic cation is tetramethylammonium.

92. The method of claim 87, wherein X is hydrogen.

93. The method of claim 87, wherein X is sodium, potassium, or ammonium.

94. The method of claim 87, wherein X is calcium or magnesium.

95. The method of claim 87, wherein X is tetramethylammonium.

96. The method of claim 87, wherein Z is hydrogen.

97. The method of claim 87, wherein Z is sodium or potassium.

98. The method of claim 87, wherein Z is calcium or magnesium.

99. The method of claim 87, wherein X is hydrogen and Z is hydrogen.

100. The method of claim 87, wherein X is sodium and Z is hydrogen.

101. The method of claim 87, wherein X is hydrogen and Z is sodium.

102. The method of claim 87, wherein X is sodium and Z is sodium.
